(19) United States
(12) Patent Application Publication
(10) Pub. No.: US 2004/0157304 A1
(43) Pub. Date: Aug. 12, 2004

(54) Molecular rotary nanomotor and methods of use

(75) Inventor: Peixuan Guo, West Lafayette, IN (US)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN (US)

(21) Appl. No.: 10/699,715

(22) Filed: Nov. 3, 2003

Related U.S. Application Data
(63) Continuation-in-part of application No. 10/660,132, now abandoned.
(60) Provisional application No. 60/411,808, filed on Sep. 18, 2002.

(51) Int. Cl. C12N 9/00

(52) U.S. Cl. 435/183

(57) ABSTRACT

A molecular rotary nanomotor useful for translocating polynucleotides. The nanomotor is a multimolecular complex fueled by ATP hydrolysis. One of the motor components is an ATP-binding RNA molecule that participates in ATPase activity.

This continuation-in-part application claims priority to U.S. patent application Ser. No. 10/660,132, filed Sep. 11, 2003, which claims the benefit of U.S. Provisional Patent Application Serial No. 60/411,808, filed 18 Sep. 2002, each of which is incorporated herein by reference in its entirety.

This application further fully incorporates by reference international patent publication PCT WO 02/16596, published Feb. 28, 2002.

### STATEMENT OF GOVERNMENT RIGHTS

This invention was made with government support under grants from the National Institutes of Health, Grant Nos. GM59944, and GM60529, and from the National Science Foundation, Grant No. MCB9723923. The U.S. Government has certain rights in this invention.

### BACKGROUND OF THE INVENTION

In living systems, cellular components are actively transported by molecular motors such as F1-ATPase, kinesin, myosin and helicase. During maturation of a DNA virus, the lengthy viral genome is translocated with remarkable velocity by a viral molecular motor into a limited space within a preformed protein shell and packaged to an almost crystalline density. Viral DNA-translocating motors includes both structural (integrated) and nonstructural (transient) components.

Bacterial virus phi29 is an unparalleled system for the study of the mechanism of DNA packaging due to its high efficiency of in vitro DNA packaging (Guo et al., 1986, Proc. Nate Acad. Sci. USA 83, 3505-3509). The phi29 DNA packaging motor has been reported to be the strongest existing molecular motor with the highest stalling force of 57 pico-newtons and a speed of 100 bases per second (Smith et al., 2001, Nature 413, 748-752). The viral motor performs the DNA packaging reaction. Neck protein gpl 1/12, tail protein gp9, and morphogenic factor gp13 are needed to complete the assembly of infectious virions. The structure of connector protein gp10 has been solved by X-ray crystallography (Simpson et al., 2000, Nature 408, 745-750; Guasch et al., 2002, J. Mol. Biol. 315, 663-676). The pRNA has been shown to form a hexamer to gear the DNA-packaging motor (Guo et al., 1998, Mol. Cell. 2, 149-155; Trottier and Guo, 1997, J. Virology, 71,487-494; Hendrix, 1998, Cell 94, 147-150; Zhang et al., 1998, Mol. Cell. 2, 141-147).

All components needed to package phi29 DNA and to assemble infectious virions have been purified and can be used for in vitro assembly of the motor. The in vitro assembly system can convert a DNA-filled capsid into an infectious virion. With this efficient system, up to 10⁸ pfu/ml of infectious virions can be assembled in vitro, while the omission of a single component results in no plaque formation (Lee et al., 1994, Virol, 202, 1039-1042; Lee et al., 1995, J. Virol. 69, 5018-5023).

The operation of a motor requires energy. In addition, to ensure the continuous motion of the motor, at least one component should act processively. In living organisms, the intriguing process of bioenergy conversion is manifest in ATP binding and hydrolysis. All bio-motors such as myosin, kinesin, DNA-helicase and RNA polymerase involve an ATP-binding component that acts processively.

ATPase activity has been long believed to be possessed by proteins only. It is generally believed, for example, that gp16 is the processive factor in driving the phi29 DNA-packaging motor. However, RNA is much easier to synthesize than proteins, and a molecular motor powered by an RNA that participates in the generation of ATPase activity would find broad use in medical and nanotechnology applications.

### SUMMARY OF THE INVENTION

The invention provides a molecular motor, termed herein a “molecular nanomotor” or simply “nanomotor,” capable of translocation of a polynucleotide. The molecular nanomotor of the invention comprises a nanoscale structure formed from the association of both protein and RNA. In one embodiment, the nanomotor is derived from a phi29 bacteriophage nanomotor and contains structural components that include a connector protein gp10, a capsid protein gp8, and a pRNA, or their equivalents. These structural components together form a nanoscale structure capable of effecting translocation of a polynucleotide in the presence of a gp16 protein, ATP and Mg⁺⁺. Optionally, protein gp7 can be included in the nanomotor as a structural component.

Two other components of the nanomotor, a gp 16 protein and ATP, are considered “nonstructural.” Although they are not structurally integrated into the nanomotor, these components impart functionality to the nanomotor. These nonstructural components are transiently associated with the structural part (i.e., the nanoscale structure) of the nanomotor. In order for the nanomotor to function, the nanomotor should be supplied with gp16, ATP and magnesium (Mg⁺⁺). An optional nonstructural component which is expected to enhance the function of the nanomotor is polyethyleneglycol (PEG), which enhances the solubility of gp16.

Translocation activity of the nanomotor can be reversibly halted by contacting the nanomotor with a chelating agent, contacting the nanomotor with a nonhydrolyzable ATP analogue, or depriving the nanomotor of a source of gp16 protein, ATP and/or Mg⁺⁺. Activity resumes when the nanomotor is supplied with additional Mg⁺⁺, ATP, or gp16 protein, depending on the method used to reversibly stop the nanomotor. Translocation activity of the nanomotor can be irreversibly stopped by contacting the nanomotor with RNase, which degrades the pRNA component.

The invention provides a method for translocating a polynucleotide that involves providing a molecular nanomotor having a nanoscale structure according to the invention, and contacting the nanoscale structure with a gp16 protein, ATP, Mg⁺⁺ and, optionally, PEG, under conditions effective to translocate the polynucleotide. Optionally, the method includes reversibly stopping the nanomotor, for example by contacting the nanoscale structure with a metal chelating agent such as EDTA or a nonhydrolyzable ATP analogue such as γ-S-ATP. The nanomotor can then be restarted as described above. The nanomotor may be irreversibly stopped by contacting it with RNase.

The nanomotor of the invention exhibits many important and unusual characteristics. For example, the nanomotor is a rotational (rotary) motor (FIG. 18). RNA serves together with proteins as a motor component, resulting in a composite RNA-protein motor structure. The rotary nanomotor contains a 6 “pole” rotor element formed from pRNAs, the connector, and gp 16 protein, and a 5-“pole” stator element made by the procapsid. The rotation of the nanomotor is counterclockwise when viewed from the portal side, suggesting that DNA packaging is achieved by utilizing the “threaded” helical nature of dsDNA. The “differential” effect of this rotary motor is due to the symmetry mismatch between the “rotor” and the “stator” of the packaging motor.

Significantly, in this unique motor the RNA component binds ATP and is part thus of the ATPase activity, thereby being involved in providing fuel to the motor. Synthetic pRNA as well as naturally occurring pRNA can be utilized, as described in more detail below. Surprisingly, the ATP-binding RNA (whether naturally occurring or synthetic, as described more fully below) has the ability to drive the nanomotor. The pRNA can be manipulated and controlled at will to form dimers and trimers with different shapes and sizes (FIG. 19) and can be derivatized with groups for linking to other components during the construction of the macromolecular complex. Therefore, the pRNA molecules could serve as ideal building blocks in nanotechnology.

The nanomotor's basic function of translocating a polynucleotide from one location to another gives it utility in a broad spectrum of scientific and industrial applications. It can, for example, be used as a nanodevice for drug delivery, delivery of genes for therapy, or the repair of chromosomes. It can be embedded in a membrane or matrix material and serve generally as a portal for translocating polynucleotides from side to the other, as in applications that require moving polynucleotides from one chamber to another.

The nanomotor can also be used to perform a sorting function. Advantageously, the 3′ end of the pRNA can be extended by up to about 120 nucleotides without affect pRNA folding and function. The extended sequence can be selected so that it provides as complementary signal to specifically pick up a polynucleotide substrate for sorting. For example, a substrate DNA or RNA can be selected by based on hybridization to the extended pRNA sequence. The selected polynucleotide is then positioned for translocation by the nanomotor. Since there are six pRNA for each complex, it would be possible to sort up to six different substrates by annealing and denaturation.

Importantly, the nanomotor functions as a molecular pump, which could have a variety of applications in clinical medicine and drug development. Moreover, as a result of its nanoscale size and weight, the nanomotor of the invention is expected to serve as the basis for the development of very strong and light novel materials including nanocomposites, small mechanical devices, and self-assembled biomaterials.

### BRIEF DESCRIPTION OF THE DRAWING

FIG. 1 is a graphical representation of the phi29 DNA packaging motor. Panels A and B show the 3D structure of the nanomotor with bottom view and side view, respectively. Panel C is a space filling model of the structure of aptRNA predicted by computer modeling based on experimental data derived from photo-affinity cross-linking, chemical modification and chemical modification interference, complementary modification, nuclease probing, and cryo-AFM.

FIG. 2 shows the use of the poorly hydrolysable ATP analogue γ-S-ATP to halt the motor and produce DNA-packaging intermediates. It also shows that the halted motor can be restored to function, since the intermediates can be converted into infectious virus after the addition of ATP.

FIG. 3 is a graph demonstrating the requirement of pRNA and gp16 for the initiation of DNA packaging by conversion of phi29 DNA-packaging intermediates into infectious virion. “Omit gp16” or “omit pRNA” indicates that during the first DNA packaging step, either gp16 or pRNA, respectively, was omitted from the DNA packaging mixture. “Complete” indicates the complete insertion of the entire genomic DNA into the protein shell. The incomplete DNA-packaging intermediates in each fraction of the gradient were subsequently converted into infectious phi29 virion by the addition of fresh gp16, ATP, neck protein gpl 1/12, and tail protein gp9.

FIG. 4 shows graphs demonstrating the requirement of fresh gp16 and ATP but no requirement of pRNA for the motor to continue and complete the DNA packaging of intermediates. Each fraction of the gradient containing DNA-packaging intermediates were subsequently converted into infectious phi29 virion in the absence of (a) pRNA; (b) gp16; (c) ATP; or (d) in the presence of RNase to cleave the pRNA in the intermediates.

FIG. 5 shows a schematic representation of the structure of phi29 DNA packaging nanomotor (Hoeprich and Guo, J. Biol Chem, 277,20794-20803, 2002). A. Computer model of three-dimensional structure of phi29 pRNA monomer based on experimental data derived from photo-affinity cross-linking; chemical modification and chemical modification interference; complementary modification; nuclease probing; and cryo-AFM. B. pRNA hexamer docking with the connector crystal structure that has a 3.6 nm central channel for DNA entry during packaging (Simpson et al., 2000, Nature 408, 745-750). Six pRNA molecules are linked by hand-in-hand interaction via the right hand loop and left hand loop.

FIG. 6 shows ATP-binding assay with ATP-agarose affinity column. A: Binding of pRNA_{wt} (A-I) and aptRNA (A-II) to ATP (Shu and Guo, J. Biol Chem, 278, 7119-7225, 2003). B. Binding of pRNA_{wt} to ATP-affinity column and elution with ADP or GTP (B-I), as well as UTP or CTP (B-II). Each insert in B shows the entire spectrum of the elution profile.

FIG. 7 shows a comparison of the central region of pRNA with the ATP-binding RNA aptamer. A. Sequence comparison of the (a) central region (SEQ ID NO:1) of pRNA_{wt}(SEQ ID NO:2) (Guo et al., 1987, Nucleic Acids Res. 15, 7081-7090; Bailey et al., 1990, J. Biol. Chem. 265, 22365-22370) with (b) the 40-base ATP-binding RNA aptamer, ATP-40-1 (SEQ ID NO:3) (Sassanfar et al., 1993, Nature 364, 550-553; Cech et al., 1996, RNA 2, 625-627). The similar bases are in lower case letters. G^{con} is a conserved base essential for ATP-binding (Shu and Guo, J. Biol Chem, 278, 7119-7225, 2003). B. Structure comparison of (a) the central region of pRNA with (b) the ATP-binding RNA aptamer. The 3D structures in c and d are derived from computer modeling and NMR (Dieckmann et al., 1996, RNA 2, 628-640), respectively. The 5′ and 3′-ends of the moiety in c and d are marked. The adenosine residue is marked (Dieckmann et al., 1996, RNA 2, 628-640). C. Sequence of the chimeric aptRNA and related mutant aptG^{con}C (SEQ ID NO:4) (see PCT WO 02/16596). D. Comparison of concentration requirement between chimeric aptRNA and wild type pRNA_{wt} in phi29 assembly E. Sequences of pRNAs from (a) SF5, (SEQ ID NO: 5), (b) B103, (SEQ ID NO: 6), (c) phi29/PZA, (SEQ ID No: 2), (d) M2/NF, (SEQ ID NO: 7), and (e) GA1, (SEQ ID NO: 8); the 3′ ends have been extended to facilitate recombinant expression.

FIG. 8 shows in vitro production of infectious virions of phi29 particles with aptRNA and ATP (Shu and Guo, J. Biol Chem, 278, 7119-7225, 2003). A. Electron microscopy (EM) image (×90,000) of purified phi29 procapsid devoid of genomic DNA. B. Plaques formed on a lawn of Bacillus subtilis after plating with the infectious virus produced from the reaction with aptRNA and ATP. C. EM image (×90,000) of the viral particles purified from the lawn in B. D. Agarose gel showing EcoRI restriction mapping of genomic DNA from wild-type phi29 (lane b), and from the virus assembled with aptRNA (lane c). Lane d shows 1-kb ladder and lane a contains a control sample from procapsid (A) that is devoid of genomic DNA.

FIG. 9 shows ATP binding affinity of pRNA and aptRNA (Shu and Guo, J. Biol Chem, 278, 7119-7225, 2003). A-B. [³H]aptRNA (A) or [³H]pRNA_{wt}, (B) was applied to a column (0.55 cm in diameter) packed with ATP-C-8 affinity agarose (0.8 ml) and eluted with a 2 ml step-up gradient with specified concentration of ATP in binding buffer. Fractions were collected and subjected to scintillation counting. C. [³H]aptRNA was applied onto a 0.8 ml ATP-agarose affinity column and washed with binding buffer, then eluted with buffer containing 0.004 mM of ADP (C-I), UTP (C-II), CTP (C-II) or GTP (C-II), then with 0.004 mM ATP. Arrows indicate that the given concentration of specified nucleotides was added to the binding buffer. Each fraction is 250 μl.

FIG. 10 shows sequences of wild type and mutant pRNAs used in confirmation verification studies (Shu and Guo, J. Biol Chem, 278, 7119-7225, 2003). The left panel (A-D) (SEQ ID NOs: 2, 9, 10 and 11, respectively) is a set of deletion mutants derived from the wild type parental pRNAW_{wt}, to confirm the conformation of mutants with a change of G^{con} (in A, B and C) to C (in D). The right panel (E, F, G and H) (SEQ ID NOs: 4, 4, 12 and 13, respectively) is a set of deletion mutants derived from parental aptRNA to confirm the conformation of mutant with a change of G^{con} (E and G) to C (F and H). The plot of (I) shows a competitive inhibition assay to compare the conformation of pRNA with and without the mutation of G^{con .}

FIG. 11 is a native gel electropherogram depicting the interaction of ATP-binding RNA with ATP. Lane a, 5S rRNA, no ATP; lanes b-c, 5S rRNA, increasing amounts of ATP; lane d, DNA ladder; lane e, aptRNA, no ATP; lanes f-h, aptRNA, increasing amounts of ATP (Shu and Guo, J. Biol Chem, 278, 7119-7225, 2003).

FIG. 12 is an autoradiogram of an ATPase assay by thin layer chromatography showing the hydrolysis of [y-³²P]ATP in the presence of pRNA (Shu and Guo, J. Biol Chem, 278, 7119-7225, 2003).

FIG. 13 depicts the results ATP-binding assay with ATP-agarose affinity column. A. Binding of aptRNA (∘); aptGconC (▪); and 116-base rRNA control (▴) to ATP-agarose affinity column. B. Elution of aptRNA from the column using ADP (∘) and ATP (▴). C. Elution of aptRNA using UTP (♦); CTP (▪); GTP (∘); and ATP (▴)(Shu and Guo, J. Biol Chem, 278, 7119-7225, 2003).

FIG. 14 depicts reversibility of motor function. The motor shut off by γ-S-ATP could be turned-on again by ATP. ATP, gp16, gp 11/12 and gp9 were added to each fraction from the sucrose gradient containing DNA-packaging intermediates, which were blocked by γ-S-ATP, and assayed for the production of infectious virus. When ATP was added (∘), viruses were produced. However, in the absence of ATP (▪), or when EDTA (▴) or RNase (X) were added, no viruses were produced, indicating that EDTA and RNase blocked the motor.

FIG. 15 depicts the passive release of DNA from protein complex. A. At pH 4 (lane c), but not pH 7 (lane b), phi29 DNA was released from the protein shell and was sensitive to EcoRI digestion, similar to purified phi29 DNA (lane d). B. Electron micrograph shows the released DNA (arrow), with TMV virus (a bar at the top) as size control.

FIG. 16 depicts the formation of ordered structural arrays. I. Formation of tetragonal arrays with the purified connector in 3M NaCl (Lee and Guo, J. Virology, 69, 5024-5032, 1995; Jimenez et al., Science 232:1113-1115, 1986;). A is an electron microscope (EM) micrograph from negative stained electronic microscopy; each unit of the connector is 14 nm wide. B is a drawing, and C is composed of the 3D crystal structures of the connector. D is the side view. II. Formation of ordered pentagonal structure. E is the EM micrograph of the purified pRNA/connector complex (Garver and Guo, RNA 3, 1068-1079,1997). F is the computer model of the 3D structure of the complex containing five connectors with pRNA hexamer located at the center of the complex. III. Hexagonal arrays containing the phi29 necks composed of connector and gp 11. G shows the hexagonal complex consisting of connector and gp 11 (Jimenez et al, Science 232:1113-1115, 1986; Carazo et al., J Mol Biol 192:853-867, 1986). H and I depict the hexagonal arrangement of the connector/gp11 complex. J is the computer model of the 3D structure of the hexagonal array shown in G.

FIG. 17 depicts binding experiments used to determine the apparent dissociation constant K_{D,app} for RNA/ATP interaction. A. Isocratic elution for ATP that was immobilized on agarose (ATP_{bound}); B. ATP gradient elution for free ATP (ATP_{free}) (Shu and Guo, J. Biol Chem, 278, 7119-7225, 2003).

FIG. 18 depicts the sequential action of pRNAs in a phi29 DNA packaging motor. The leftmost image is the three-dimensional structure of the motor complex including the connector and pRNA hexamer. In A-G showing the six steps of rotation, the hexagon represents the phi29 connector and the surrounding pentagon represents the capsid. Six protrusions represent six pRNAs with variable pRNA patterns portraying the pRNA in serial energetic states. For example, pRNA 4 and 1 in panel A represent contracted and relaxed conformations, respectively. Arrows mark the different transition states of pRNA 1. Each step, e.g., A to B, rotates 12°, since a five to six-fold symmetry mismatch generates 30 equivalent positions, and 360°/30=12°. The portal vertex turns 72° after six steps of rotation. For example, pRNA 1 moves from vertex a in A to vertex b in G, and rotates 72°. Each step consumes one ATP to induce one conformation change of pRNA, and six ATPs are used for the transition from one vertex to another.

FIG. 19 illustrates the formation of pRNA dimers and trimers with variable shapes. Dimers and trimers that have all of the left and right hand loops bound via hand-in-hand interactions are called “closed”. Dimers and trimers that have one left and one right hand loops not bound via hand-in-hand interactions are called “open”. Open dimers and trimers are shown with an “X” between the left and right hand loops that do not base pair. Cartoons illustrate the concept of hand-in-hand interactions. The right column is the direct observation of purified monomer, dimer and trimer with Cryo-AFM (Atomic Force Microscope). RNA monomers, dimers and trimers with variable lengths are likely to be useful in the construction of nanodevices.

### DETAILED DESCRIPTION OF ILLUSTRATIVE EMBODIMENTS

The construction of nanoscale artificial motors by chemical synthesis is an intriguing endeavor in contemporary technology. We show here that a 30-nanometer motor can be made in vitro with purified recombinant proteins and artificially designed RNAs.

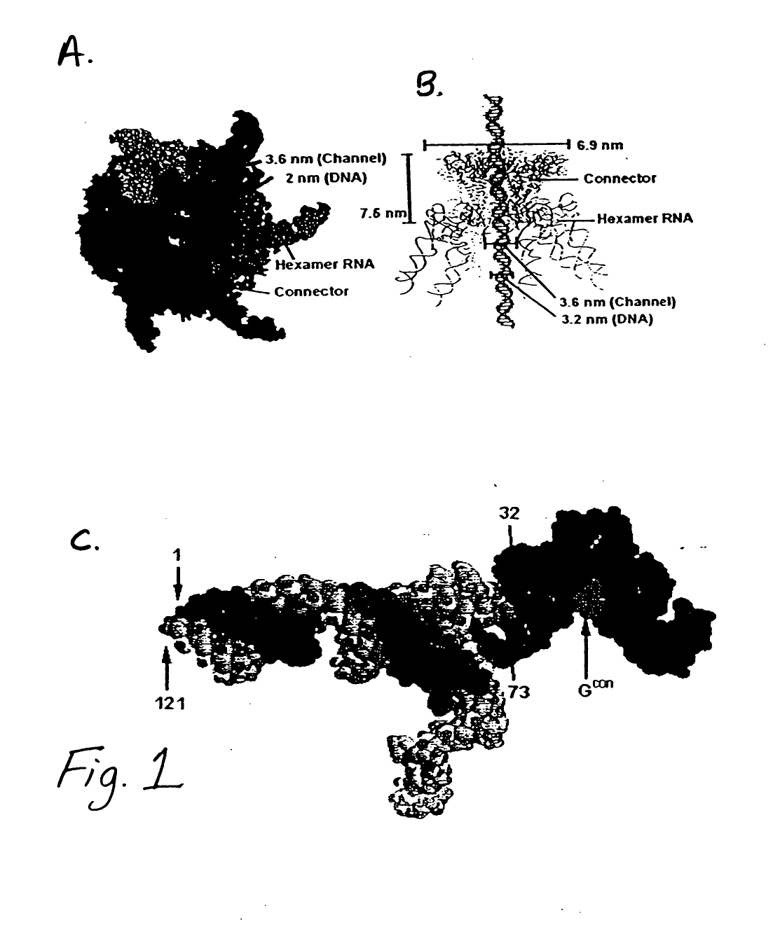

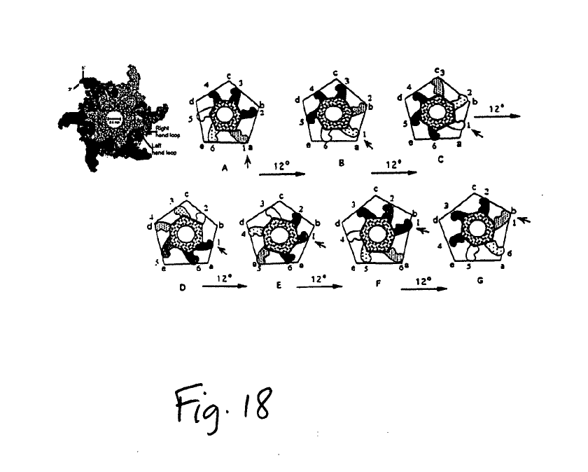

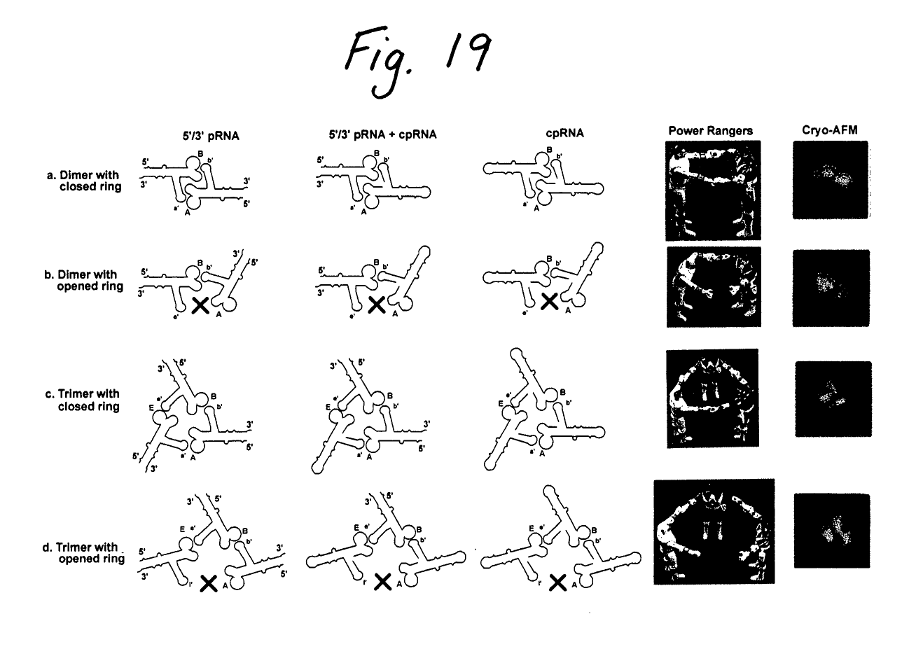

The 30-nanomotor exemplified herein is modeled on the sequential action of pRNA in phi29 DNA packaging (FIGS. 1, 18, and 19). Phi29 contains a capsid with a five-fold symmetry. Interaction of the hexamer RNA and the capsid generates a five to six-fold symmetrical match to facilitate a continuous rotation of the motor. Each step rotates 120, since a five to six-fold mismatch generates 30 equivalent orientations (360°/30=12°). The variable shapes and patterns portray six RNA in serial energetic states. Each 12° rotation will move one of six RNA to align with one vertex of the pentagon. The portal vertex turns 72° after six steps. For example, RNA #2 moves 12° from panel A to touching the vertex b in panel B. In A, RNA #1 is aligned with vertex a, while in B, RNA #1 is 12° away from vertex a. Each 12° increment consumes one ATP. Therefore, 30 ATPs are needed for one 360° rotation.

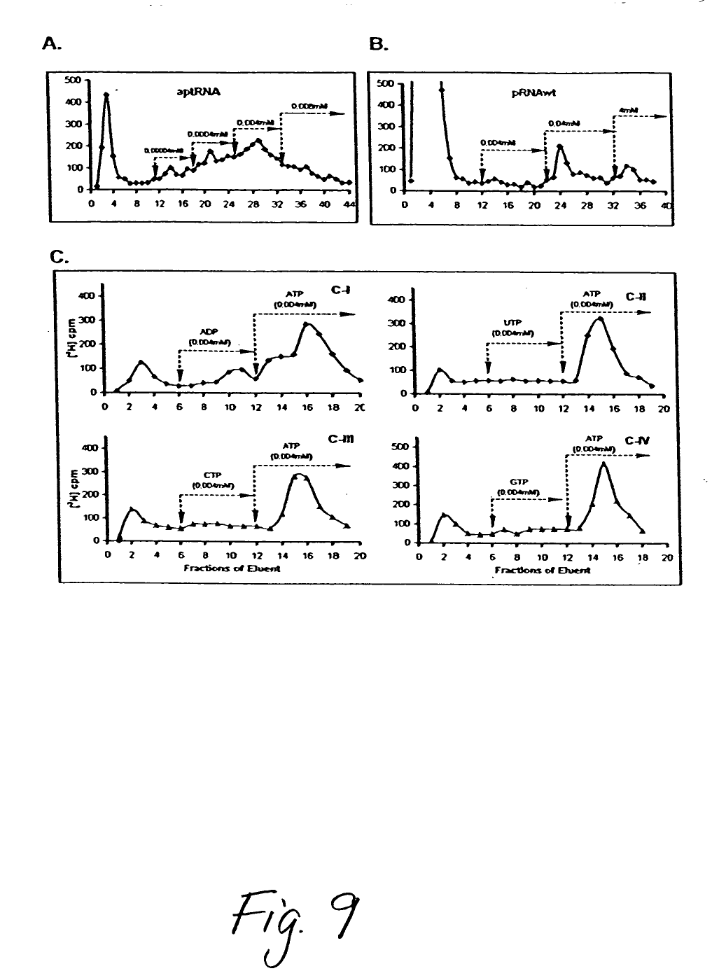

ATP-binding RNA, dubbed aptamer, was identified from synthesized random RNA pools using a chemical in vitro selection and amplification technique. A 40-base RNA aptamer was selected chemically and found to be able to bind ATP. Using this 40-base ATP-binding RNA aptamer as a central element (FIG. 7A(a)), a chimeric 121-base pRNA, called aptRNA, was constructed (FIG. 7C) to imitate the DNA-packaging pRNA of bacterial virus phi29. Amazingly, this aptRNA was able to power the protein complex to pump the viral DNA genome into the protein shell, and to produce infectious virus in the test tube. ATP is used as the source of energy. The mechanics of the motor resemble the driving of a bolt with a hex nut with six pRNAs forming a hexagonal complex to gear the DNA translocating machine in 12° increments.

Importantly, the processive factor in the phi29 DNA-packaging motor was discovered to be the pRNA not gp16. The pRNA is a structural part of the nanomotor and also acts as an enzyme, constantly working. The protein gp16, on the other hand, appears to be transiently associated with the complex, although it is, nonetheless, apparently required for the first round of assembly, and needs replenishment if the motor is to function; it is a transient distributive factor in motor function. For the nanomotor to function, a continuous supply of gp16, ATP and Mg⁺⁺is needed.

The molecular nanomotor can be reversibly turned off by the addition of a nonhydrolyzable ATP analog, e.g., γ-S-ATP or a metal chelating agent, such as EDTA. If a nonhydrolyzable ATP analog is used to turn off the nanomotor, it can be restarted by adding ATP. If EDTA or other chelating agent is used to turn off the nanomotor, the addition of magnesium will restart it. The nanomotor can also be reversibly turned off by depriving the nanomotor of the distributive factor, gp16, or depriving it of ATP, thereby eliminating the fuel source. The nanomotor can be restarted with the addition of fresh gp16 or ATP, respectively. Irreversible shut-down of the nanomotor can be accomplished by treating the nanomotor with RNase, which compromises its structural integrity by degrading the pRNA component.

Component Proteins

The proteins described herein for use as components of the molecular nanomotor can include naturally occurring or synthetic sequences. In other words, although a preferred embodiment of the nanomotor utilizes protein components in their naturally occurring form, proteins that are structurally and functionally equivalent can be used. Unless otherwise indicated herein, when a structural or nonstructural protein component of the nanomotor, such as “protein gp16” is referred to herein, that term includes proteins that are both structurally and functionally equivalent to the protein referred to. The proteins used as components of the nanomotor can be isolated directly from bacteriophage, produced recombinantly, or enzymatically or chemically synthesized.

Structural equivalency can be defined by reference to the level of amino acid identity between the sequence of the candidate protein used in the nanomotor and the corresponding reference, naturally occurring sequence. Preferably, a structurally equivalent protein has an amino acid sequence that shares at least an 80% amino acid identity to the corresponding naturally occurring sequence. Amino acid identity is defined in the context of a homology comparison between the candidate sequence and the reference sequence. The two amino acid sequences are aligned in a way that maximizes the number of amino acids that they have in common along the lengths of their sequences; gaps in either or both sequences are permitted in making the alignment in order to maximize the number of shared amino acids, although the amino acids in each sequence must nonetheless remain in their proper order. The percentage amino acid identity is the higher of the following two numbers: (a) the number of amino acids that the two polypeptides have in common within the alignment, divided by the number of amino acids in the candidate protein, multiplied by 100; or (b) the number of amino acids that the two polypeptides have in common within the alignment, divided by the number of amino acids in the reference protein, multiplied by 100. It should be understood that structural equivalents of a protein can included derivatives of a protein (e.g., proteins that have been altered by amidation, acetylation and the like) as well as proteins having deletions or additions with respect to the reference protein (e.g., truncated proteins).

Functional equivalency of a candidate protein is defined as retention of at least a portion of the reference protein's binding or enzymatic activity. Structural proteinaceous components of the nanomotor should retain an ability to associate with (bind) other structural components of the nanomotor. Nonstructural proteinaceous components of the nanomotor should retain an ability to transiently associate with the nanomotor structure and should exhibit at least a portion of the protein's enzymatic activity (e.g., in the case of gp16, the ability to perform the distributive function). The binding and/or enzymatic activity of the various proteins used as components in the nanomotor described herein can be readily determined by evaluating the efficacy of DNA packaging and/or viral assembly assay as set forth in detail in the Examples below.

One of skill in the art of protein biochemistry will appreciate that there are a number of conservative changes that can be made to the amino acid sequence of the reference protein without significantly altering its binding characteristics or other activity. These changes are termed “conservative” mutations, that is, an amino acid belonging to a grouping of amino acids having a particular size or characteristic can be substituted for another amino acid, particularly in regions of the protein that are not associated with catalytic activity or binding activity, for example. Substitutes for an amino acid sequence may be selected from other members of the class to which the amino acid belongs. For example, the nonpolar (hydrophobic) amino acids include alanine, leucine, isoleucine, valine, proline, phenylalanine, tryptophan, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Particularly preferred conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free NH₂.

Component pRNA

The nanomotor requires, as a structural component, a pRNA molecule that binds ATP. The pRNA molecule contains a central ATP binding region, flanked by binding regions that facilitate association of the RNA to the other structural components to form the nanomotor structure. In a preferred embodiment, the flanking regions contain ribonucleotides 1-32 and 69-117 of naturally occurring phi29 RNA (FIG. 7). However, the specific sequence of pRNA is not critical; the important feature of the pRNA is that the secondary and tertiary (3D) structures are similar to native pRNA, allowing the pRNA to bind phi29 procapsid.

The central region involved in ATP binding comprises ribonucleotides 33-68, and it has been found that these nucleotides can be substituted with another ATP binding sequence without affecting motor function (Shu and Guo, J. Biol Chem, 278, 7119-7225, 2003). Additional ribonucleotides, whether or not derived from naturally occurring phi29 pRNA, can be attached to the 5′ and 3′ ends of the pRNA. As noted above, it has been found that up to about 120 ribonucleotides, and maybe more, can be attached to the 3″end of the pRNA without affecting pRNA folding and function. Thus, the pRNA component of the nanomotor can include naturally occurring or synthetic ribonucleotide sequences. It has been surprisingly found that non-naturally occurring pRNA (e.g., a chimeric pRNA containing aptRNA, as described below, and pRNAs described in Chen et al. (1999, RNA 5, 805-818), Zhang et al. (1994, Virol. 201, 77-85) and Zhang et al. (1997, RNA 3, 315-322) and FIG. 7, can function in the nanomotor. Thus, pRNAs that are structurally and functionally equivalent to native bacteriophage phi29 pRNA can be used in the nanomotor. Unless otherwise indicated herein, when pRNA is referred to herein as a structural component of the nanomotor, that term includes RNAs that are structurally and functionally equivalent to phi29 pRNA.

Structural equivalency can be defined by reference to the level of ribonucleotide identity between the sequence of the candidate pRNA used in the nanomotor and a reference pRNA sequence, such as that derived from bacteriophage phi29. The regions that flank the central, ATP binding region of the candidate pRNA are preferably at least 60% identical to, more preferably 80% identical to, even more preferably 90% identical to, and most preferably 95% identical to the corresponding ribonucleotide sequence of native phi29 pRNA or the pRNA sequence of pRNA (SEQ ID NO: 2) sequences derived from phage SF5 (SEQ ID NO: 5), B103 (SEQ ID NO: 6), M2/NF (SEQ ID NO: 7) or GA1 (SEQ ID NO: 8) which exhibit the same secondary tertiary structure as phi29 pRNA (see FIG. 7). Percent identity is determined by aligning two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. For example, the two nucleotide sequences are readily compared using the Blastn program of the BLAST 2 search algorithm, as described by Tatusova et al. (FEMS Microbiol Lett 1999, 174:247-250). Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. In addition or alternatively, the pRNA used in the nanomotor contains at least 8, more preferably at least 15, most preferably at least 30 consecutive ribonucleotides found in native phi29 pRNA. In the central region of the pRNA, structural equivalence to phi29 pRNA is desirable but not required.

Functional equivalency of a candidate pRNA is defined as retention of at least a portion of the ability to bind ATP, and to associate with the structural proteinaceous components of the nanomotor to form a nanomotor structure with ATPase activity. ATP binding activity is preferably found in the central region of the pRNA. In the motor, gp16 together with pRNA form a functional hexameric ATPase. It should be noted that, for ATP binding activity to be retained, nucleotide G^{con} (FIG. 7) should be retained.

### EXAMPLES

The present invention is illustrated by the following examples. It is to be understood that the particular examples, materials, amounts, and procedures are to be interpreted broadly in accordance with the scope and spirit of the invention as set forth herein.

### Example I

### Processive Action of pRNA Drives Bacterial Virus phi29 DNA-Packaging Motor

Materials AND Methods

Preparation of pRNA

RNAs were prepared as described in Zhang et al. (1994, Virol. 201, 77-85). Briefly, DNA oligonucleotides were synthesized with the desired sequences and used to produce double-stranded DNA by PCR. The DNA products containing the T7 promoter were cloned into plasmids. RNA was synthesized with T7 RNA polymerase by run-off transcription and purified from a polyacrylamide gel. The sequences of both plasmids and PCR products were confirmed by DNA sequencing.

In Vitro Production of Infectious Virions of phi29 Virion Particles With aptRNA and ATP

The purification of procapsids (Bjornsti et al., 1985, J. Virol. 53(3), 858-861; Vinuela et al., 1976, Philosophical Transactions of the Royal Society of London-Series B: Biological Sciences 276, 29-35), gp16 (Guo et al., 1986, Proc. Natl. Acad. Sci. USA 83, 3505-3509) and DNA-gp3 (Ortín et al., 1971, Nature New Biol. 234, 275-277), the preparation of the tail protein (gp9) (Garcia et al., 1983, Virology 125, 18-30; Lee et al., 1995, J. Virol. 69, 5018-5023) neck proteins (gpl 1, gp12) (Carrascosa et al., 1974, FEBS Lett. 44(3), 317-321) the morphogenetic factor (gp13) (Lee et al., 1995, J. Virol. 69, 5018-5023), and the procedure for the assembly of infectious phi29 virion in vitro (Bjornsti et al., 1982, J. Virol. 41, 408-517; (Lee et al., 1995, J. Virol. 69, 5018-5023) were accomplished as previously described.

Briefly, 1 μg of pRNA or its active derivatives (Chen et al., 1999, RNA 5, 805-818; Zhang et al., 1994, Virol. 201, 77-85; Zhang et al., 1997, RNA 3, 315-322), in 1 μl RNase-free H₂O was mixed with 10 μl of purified preformed procapsids (0.4 mg/ml) that devoid of DNA (and dialyzed on a 0.025 μm type VS filter membrane against TBE (2 mM EDTA, 89 mM tris borate/pH 8.0) for 15 minutes at room temperature. The mixture was subsequently transferred for another dialysis against TMS (100 mM NaCl, 10 mM MgCl₂, 50 mM tris/pH 7.8) for an additional 30 minutes.

In the first round, the DNA packaging step, the pRNA-enriched procapsids were then mixed with gp16, DNA-gp3 (a nucleic acid/viral protein covalent chimera that facilitates the translocation of the DNA), and ATP (1.4 mM final concentration except when otherwise indicated) to complete the DNA packaging reaction.

After 30 minutes, in the second round, the assembly step, gpl 1, gp12, gp9, and gp13, and gp16 were added to the DNA packaging reactions to complete the assembly of infectious virions, which were assayed by standard plaque formation.

Isolation of DNA-Packaging Intermediates and Conversion of the Intermediates Into Infectious phi29 Virion

A poorly hydrolyzable ATP analogue, γ-S-ATP, was used in the DNA packaging step (first round) to produce DNA packaging intermediates. DNA-packaging intermediates were generated by the addition of 5% γ-S-ATP (i.e., addition of 1:20 γ-S-ATP:ATP to reach 1.4 mM ATP final concentration) into the phi29 in vitro first round DNA packaging mixture. The intermediates were separated from free DNA and the finished DNA-filled procapsids by 5-20% sucrose gradient sedimentation with SW65 rotor for 30 minutes at 35000 rpm. The gradients were fractionated to separate the components that have different sedimentation rate.

The components in each fraction of the gradient were subsequently converted into infectious phi29 virion by the addition fresh components for phi29 in vitro second round assembly. The complete conversion system (including first and second round components) includes pRNA, gp16, ATP, neck protein gpl 1/12, and tail protein gp9 and gp13. The infectious virion were titrated by plating on the bacterial host Bacillus subtilis Su⁺⁴⁴.

ATP-Binding Assay For pRNA With ATP-Agarose Affinity Column

A 0.55 cm diameter column was packed with affinity agarose resin (Sigma) immobilized with 1.25-3.25 mM ATP (or other nucleotides) and attached through the C8 (or other position) to cyanogen bromide-activated agarose. Lyophilized resin was soaked in distilled water for more than a half-hour before column packing. After washing with 10 ml of distilled water and then with 10 ml of binding buffer (300 mM NaCl, 20 mM tris/pH 7.6, 5 mM MgCl₂), 1 μg (2.5×10⁻⁵ μmole) of [³H]-labeled RNA in 100 μl binding buffer was applied to the ATP affinity column. The column was then washed with 3 ml of binding buffer, and eluted with the same buffer containing ATP or other nucleotides as indicated. Fractions were collected and subjected to scintillation counting. A 116-base rRNA was used as a negative control.

ATP Gradient Elution to Evaluate the ATP Binding Affinity of pRNA and aptRNA

In ATP gradient elution, a 0.8 cm diameter column was packed with 0.8 ml ATP C-8-agarose immobilized with 1.7 mM ATP. 1 μg (2.5×10⁻⁵ μmole) of [³H]pRNA in 100 μl binding buffer was applied to the column. After washing with 5 ml of binding buffer, RNA was eluted with a 2 ml step-up gradient with increasing concentration of ATP in binding buffer.

Verification of Mutant pRNA Conformation by Competitive Inhibition Analysis

Measurement of binding affinity and virion assembly activity is a reliable and simple method to evaluate conformational changes of mutants with mutations at the location involved in binding. Competitive inhibition assays in combination with binomial distribution were performed to determine the binding affinity. A fixed amount of parental pRNA, pRNA_{wt} or aptRNA was mixed with a varied amount of mutant competitor pRNA in a two-fold serial dilution. Parental pRNA is similar to pRNAW, except that it has two bases at the 5′ and 3′ ends changed to initiate T7 transcription. The “fixed amount” was first determined by titrating a concentration dependant curve of parental pRNA via the plotting of concentration (X-axis) of parental pRNA against the yield of procapsid/pRNA complex (if it is for procapsid binding assay) or virions assembled (if it is used for virion assembly assay). A pRNA concentration required to produce 90% of the maximum yield was taken as the fixed amount of parental pRNA in competitive inhibition analysis.

a. Conformation verification by competitive inhibition assays for procapsid binding.

5ul (2 mg/mil) of purified procapsids in TMS were dialyzed against TBE on a 0.025-um type VS filter membrane at room temperature for 15 minutes. 1 ug of [³H]-parental pRNA (pRNA_{wt} or aptRNA) was mixed with a varied amount of unlabelled competitor RNA in 3 ul of TMS and dried by vacuum. Then the RNAs were resuspended in 5 ul of procapsids that had been dialyzed against TBE for 15 minutes. As a result, the binding volume was limited to 5 ul, and the molar concentration of pRNAs was achieved at a level as high as several uM. After dialysis for another 30 minutes against TMS at room temperature, 95 uL of TMS was added to bring the volume to 100 ul, and the mixtures were then subject to sedimentation via 5-20% sucrose gradient made in TMS to separate procapsid-bound pRNAs from unbound ones. Again, the total cpm of bound [³H]-parental pRNA was plotted against the molar ratio of competitor/total pRNA.

b. Conformation verification by competitive inhibition assays for phi29 assembly and the use of binomial distribution to interpret the inhibition curve.

The procedure for using binomial distribution to predict competitive inhibition curves has been described (Trottier et al., 1997, J. Virol. 71, 487-494; Chen et al., 1999, RNA 5, 805-818; Chen et al., 1997, Nucl. Acids Sym. Ser. 36, 190-193). Briefly, in vitro phi29 assembly was performed in the presence of various ratios of parental and mutant pRNAs. The distribution probability of procapsids containing a certain number of mutant and wildtype pRNA was calculated using the binomial equation:

$${( p + q )}^{z} = ( \begin{matrix}z \\ 0\end{matrix} ) {p}^{z} + ( \begin{matrix}z \\ 1\end{matrix} ) {p}^{z - 1} q + ( \begin{matrix}z \\ 2\end{matrix} ) {p}^{z - 2} {q}^{2} + … ( \begin{matrix} z \\ z - 1\end{matrix} ) {pq}^{z - 1} + ( \begin{matrix}z \\ z\end{matrix} ) {q}^{z} = {\sum }_{M = 0}^{z} ( \begin{matrix}Z \\ M\end{matrix} ) {p}^{z - M} {q}^{M}$$

where

$$( \begin{matrix}Z \\ M\end{matrix} )$$

is equal to:

$$( \frac{Z !}{M ! ( Z - M ) !} ) ,$$

and Z represents the total number of pRNA per procapsid, while p and q represent the % of mutant and parental pRNA, respectively. Since the copy number, Z, of pRNA per procapsid is 6, the expansion of (p+q)⁶ is equal to P⁶+6P⁵q+15P⁴q²+20P³q³+15P²q⁴+6Pq⁵+q⁶. Since p and q are the known number used in assembly, the inhibition curves can be predicted as soon as the activity of parental pRNA has been determined. The probability calculation was extrapolated to predict the yield of pfu/ml produced in each in vitro phi29 assembly reaction. The curves representing the yield of virions from empirical data were plotted against the ratio of mutant pRNA/parental pRNA in the reaction and compared to a predicted curve. If the empirical curve matches the predicted curve, it is an indication that the mutant inactive pRNA had the procapsid binding affinity equal to parental pRNA, that is, the mutant did not change conformation and folding of the pRNA significantly.

ATPase Assay By Thin Layer chromatography

The purified DNA packaging components gp16 (0.24 μg), DNA-gp3 (0.1 μg), procapsid (3.2 μg) and RNA (1 μg) were mixed, individually or in combination, with 0.3 mM unlabeled ATP and 0.75 μCi (600 μCi/mmole) [γ-³²P]ATP in reaction buffer (Guo et al., 1986, Proc. Nat'l Acad. Sci. USA 83, 3505-3509). When one or more components were omitted, they were replaced with the same volume of TMS. After 30 minutes of incubation at room temperature, 3 μl of the reaction mixture was spotted on to PEI-cellulose plate (J. T. Chem. Co) (Guo et al., 1987, J Mol Biol 197, 229-236) and air-dried. The plate was then soaked in methanol for 5 minutes; air-dried and ran in 1 M formic acid and 0.5 M lithium chloride. Autoradiograms were produced with Cyclone Storage Phosphor Screen. At the same time, a parallel experiment was performed with the same components to test the results of phi29 virion assembly. Only the assembly reactions with the yield higher than 5×10⁷ plaque-forming units per milliliter were selected for ATPase assay.

Results

Isolation of DNA-Packaging Intermediates

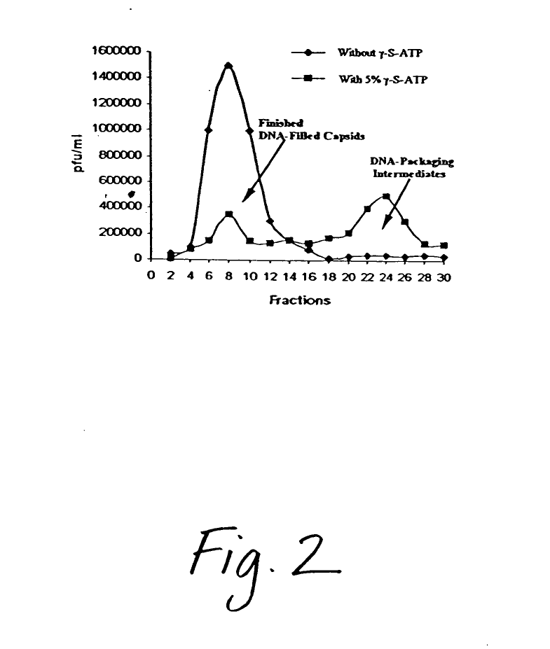

To generate DNA packaging intermediates, the poorly hydrolyzable ATP analog γ-S-ATP was used in the first round packaging reaction. Phi29 DNA packaging was performed in a mixture containing procapsid, gp16, pRNA, genomic DNA-gp3, ATP:γ-S-ATP (1:20), and magnesium. DNA packaging intermediates were separated from free DNA and finished DNA-filled capsids or empty procapsids by sucrose gradient sedimentation. The finished DNA-filled capsids centered at fraction 8 of the gradient (see FIG. 2), while smaller or lighter particles such as free DNA stayed near the top of the gradient. When 5% γ-S-ATP was included in the reaction, significant amounts of DNA-packaging intermediates with smaller sedimentation rates were produced (Fractions 22-26, FIG. 2). DNA packaging was incomplete, and a fragment of the DNA extended from the procapsid. When the ATP included in the DNA-packaging mixture did not include γ-S-ATP, very little DNA packaging intermediates were produced.

After sedimentation, the finished DNA-filled capsids and the DNA packaging intermediates in each fraction of the gradient were converted into mature infectious phi29 virions by the addition of gp16, ATP, neck protein gp 11/12, and tail protein gp9. No additional pRNA was added. The resultant infectious virions were titrated by plating on the bacterial host Bacillus subtilis Su⁺⁴⁴.

Both gp 16 and pRNA are Required for the Formation of DNA Packaging Intermediates

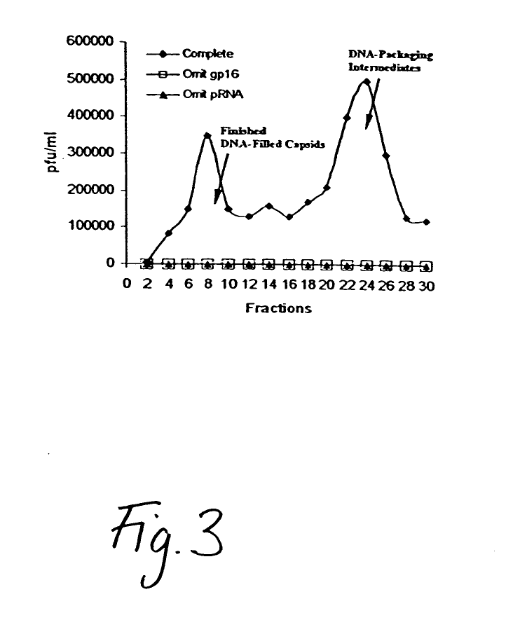

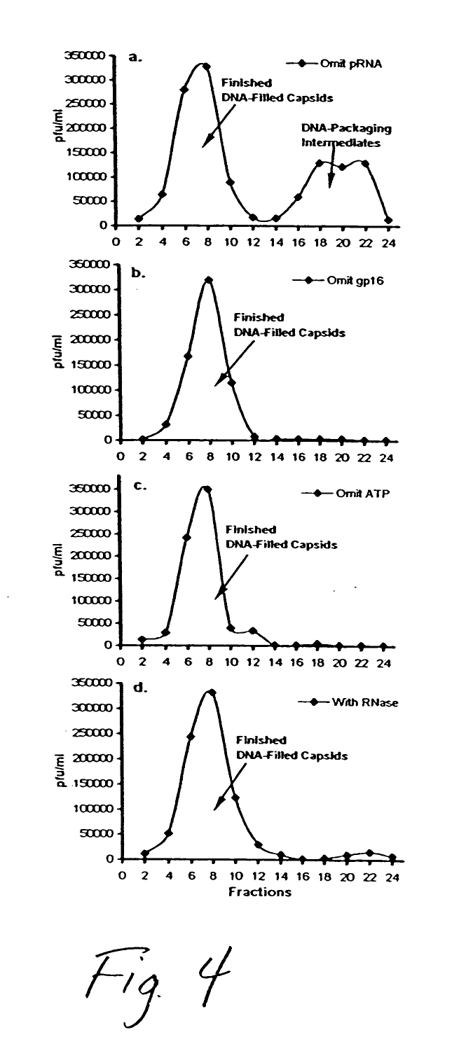

The aforementioned DNA-packaging intermediate isolation method was used to determine which components were necessary for the formation of DNA-packaging intermediates. After sucrose gradient sedimentation of first round packaging reactions including γ-S-ATP, the DNA packaging intermediates were converted in the second round into infectious phi29 virion as described above (FIG. 3). It was found that both gp16 and pRNA were needed for the formation of the intermediates in the first round DNA packaging reaction. If either gp16 or pRNA was omitted from the packaging mixture, no finished DNA-filled capsids or DNA-packaging intermediates were produced in the second round assembly (FIG. 3). Addition of Fresh gp16 and ATP Molecules to DNA-Packaging Intermediates was Required While Fresh pRNA was not Needed to Convert the DNA-Packaging Intermediates Into Finished DNA-Filled Particles

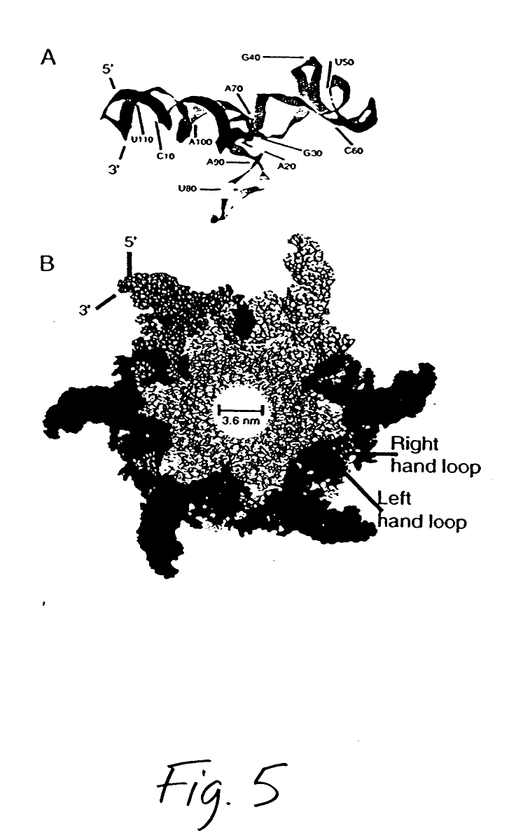

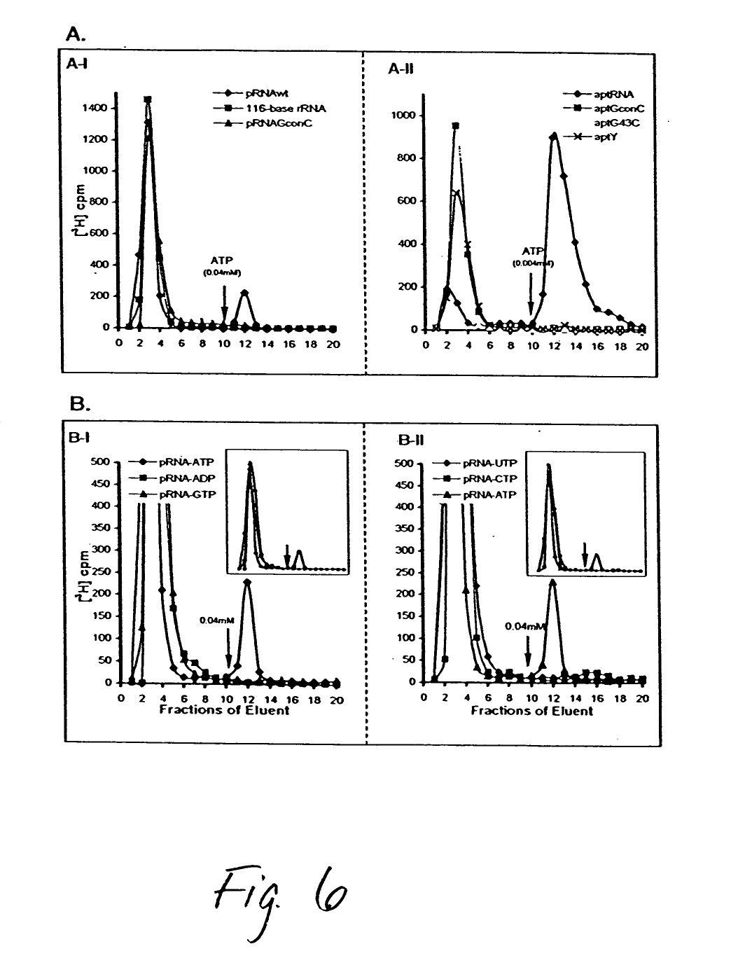

The isolated DNA-packaging intermediates produced from DNA packaging reactions using γ-S-ATP were tested to find out which components are needed to complete the packaging process. ATP, gp16, and pRNA were added individually, or in combination, into each fraction of the gradients in the presence of gp11/gp12 and gp9. It was found that it was not necessary to add pRNA to convert the finished DNA-filled capsid into infectious virion (FIG. 4a), indicating that the binding of six copies of pRNA were sufficient for the continuation of the packaging of the entire DNA genome (FIG. 5). However, it was necessary to add fresh gp16 and ATP to convert DNA-packaging intermediates into infectious phi29 virion (FIGS. 4b and 4c), indicating that the action of gp16 and ATP is not processive. That is, renewed gp16 and ATP were needed during the DNA packaging process and each gp16 and ATP molecule only played a transient role.

The Motor-Bound pRNA was Indispensable During the DNA Translocating Process

It has been reported previously that six pRNA binds to the motor (Guo et al., 1998, Mol. Cell. 2, 149-155; Trottier et al., 1997, J. Virol. 71, 487-494; Zhang et al., 1998, Mol. Cell. 2, 141-147). As already noted, it is not necessary to add fresh pRNA to complete the DNA packaging process. To test whether the procapsid bound pRNA was needed during the DNA translocating process, RNase treatment was conducted to cleave the motor-bound pRNA. It was found that after RNase treatment, the DNA-packaging intermediates could not be converted into infectious virion, while the RNase treatment did not affect the conversion of the finished DNA-filled capsid into infectious virion (FIG. 4d). This is an indication that continued function of pRNA is needed during the DNA translocating process.

Phi29 pRNA Was Able to Bind ATP

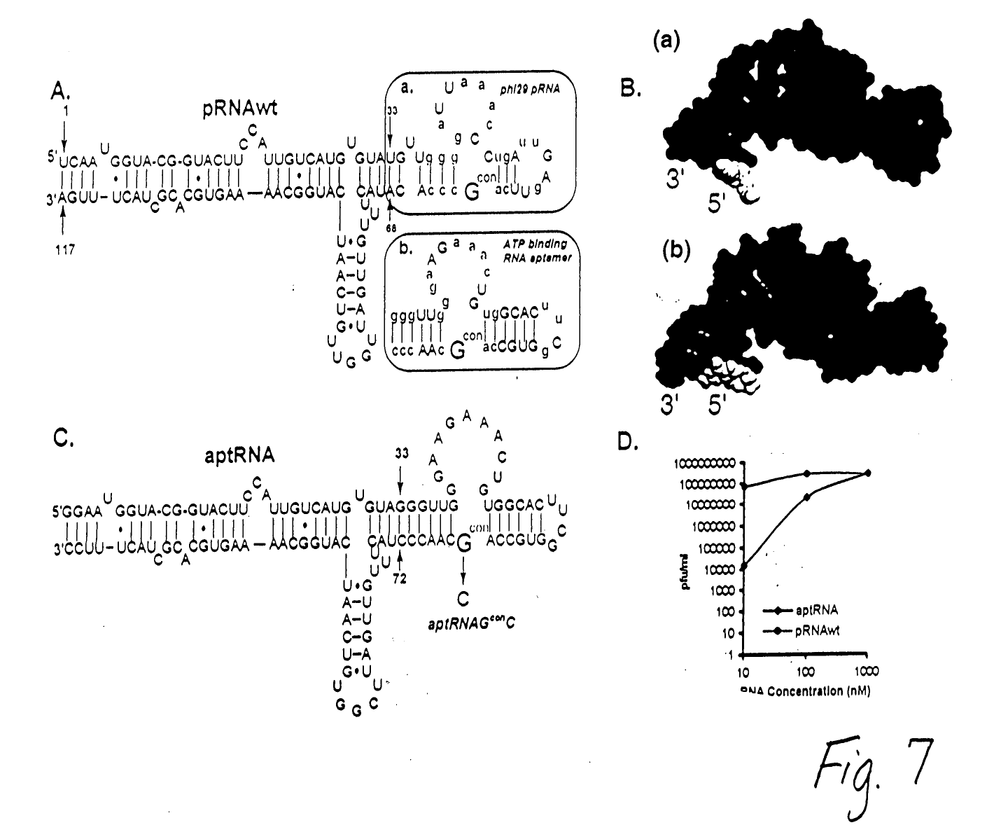

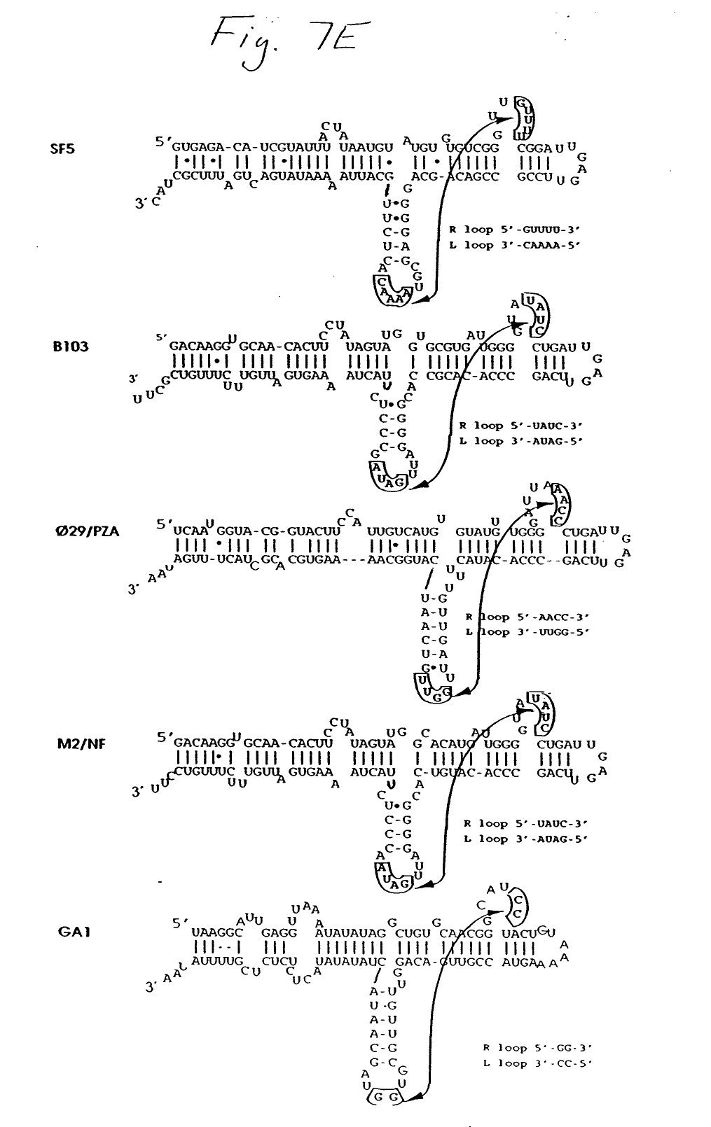

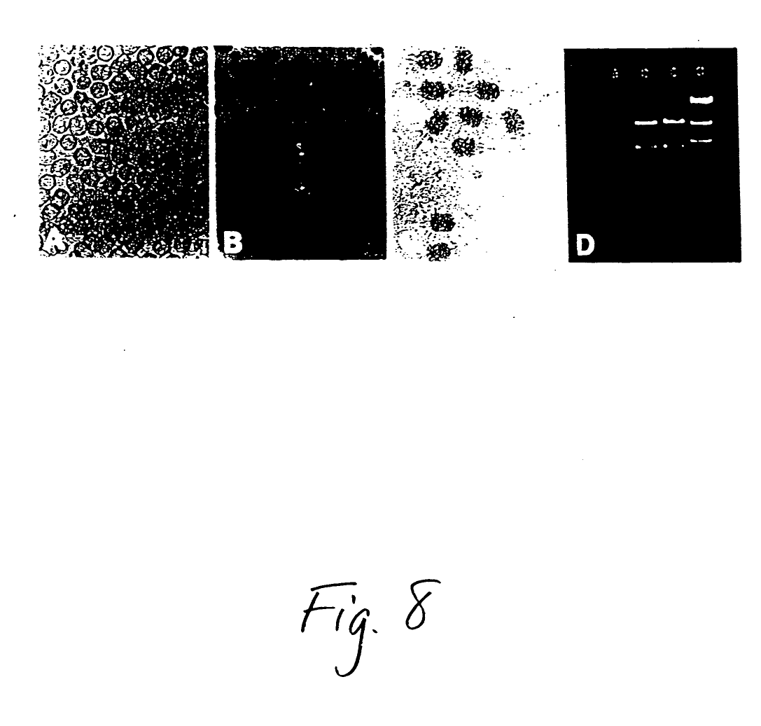

To investigate whether pRNA could interact with ATP directly, an ATP-agarose affinity column was used to detect the binding of pRNA_{wt}, the shortest pRNA with wildtype pRNA phenotype, to ATP. In FIG. 6, panel A-I, the [³H]pRNA_{wt}, mutant PRNAG^{con}C, and 116-base negative control rRNA were applied onto a 0.8 ml ATP-agarose affinity column and washed with binding buffer. After ten 250-111 fractions, the column was eluted with 0.04 mM ATP in same binding buffer. In FIG. 6, panel A-II, the [³H]aptRNA and other three mutants were tested. [³H]pRNA_{wt} eluted from the column when 0.04 mM ATP was added to the binding buffer, suggesting that pRNA_{wt} binds ATP specifically. When the 116-base rRNA served as the negative control, no detectable RNA was eluted by as high as 5 mM ATP buffer (FIGS. 6A-I), thereby indicating that the pRNA/ATP interaction was specific to pRNA. When the conserved base G^{con}, essential for ATP-binding (see below and FIG. 7A), was changed to a C, the resulting mutant pRNAG^{con}C could not bind ATP (FIGS. 6A-I) (Table 1).

TABLE 1
ATP-binding and viral assembly activities
of pRNA and mutants

| | | | Components added | | | |
|---|---|---|---|---|---|---|
| | | ATP- | | | | Virus |
| | | binding | | | γ-s- | produced |
| RNAs | Mutation | (%) | ATP | RNase | ATP | (pfu/ml) |
| aptRNA | U³³-A⁶⁸ | 80 | − | − | − | 0→ |
| | replaced | | − | − | + | 0→ |
| | by ATP | | + | + | − | 0→ |
| | aptamer | | + | − | + | 0→ |
| | | | + | − | − | 3 × 10⁸ |
| pRNA_{wt} | wild type | 20 | + | − | − | 3 × 10⁸ |
| | pRNA | | | | | |
| aptG^{con}C | G^{con}→C | 0→ | + | − | − | 0→ |
| pRNA_{wt}G^{con}C | G^{con}→C | 0→ | + | − | − | 0→ |
| 116-base | | 0→ | + | − | − | 0→ |
| rRNA | | | | | | |

ATP Binding Affinity of Resins Immobilized With Different Nucleotides or Different Linking Sites

Seven different affinity resins were tested for pRNA_{wt} binding affinity. These resins varied in nucleotide composition and in location for nucleotide/agarose linkage. Our results show that pRNA_{wt} or aptRNA bound only to an agarose resin containing ATP, but not ADP or adenosin-3′,5′-Diphosphate. For ATP resin, pRNA bound only to agarose resins with the attachment site at the C-8 position, but not at N6 or the hydroxyl position. These results suggest that the pRNA_{wt}/ATP interaction requires a specific three-dimensional configuration, and that wild type pRNA_{wt} has a much stronger binding affinity for ATP than for ADP.

Comparison of aptRNA and pRNAW_{wt} Binding Affinity to ATP and ADP

It has been reported that in the phi29 DNA packaging system, ATP is hydrolyzed to ADP during packaging (Guo et al., 1987, J Mol Biol 197, 229-236). It would be interesting to know whether pRNA_{wt} can discriminate ATP from ADP. Both ATP and ADP-affinity agarose column immobilized with ATP or ADP, respectively, and attached through the C8 position were used to compare their binding affinity for aptRNA and pRNA_{wt}. As noted earlier, both aptRNA and pRNA_{wt} could attach to ATP-affinity agarose column. However, with the ADP-affinity agarose column, aptRNA or pRNA_{wt} did not bind to the column and passed through the column, appearing only in the first several fractions of the elution. When the ADP column was eluted with 5 mM ADP or ATP, the elution of aptRNA or pRNA_{wt} from the column was almost undetectable, indicating that the binding affinity of aptRNA and pRNA_{wt} to ADP was much lower than that of ATP.

Other approaches for affinity comparison were also made. [³H]aptRNA or [³H]pRNA_{wt} were applied to the ATP-affinity agarose column first, then eluted by ATP or ADP, respectively. Comparison of the elution profiles by ATP and ADP revealed that most of the bound aptRNA and pRNA_{wt} were eluted by 0.004 mM and 0.04 mM ATP, respectively. However, in spite of an expected higher affinity for free ADP then for immobilized ADP (see above), very little aptRNA or pRNA_{wt} was eluted by ADP, even with an ADP concentration as high as 5 mM, supporting the supposition that the binding affinity of aptRNA and pRNA_{wt} to ADP was much lower than that of ATP.

Comparison of RNA Binding Affinity for ATP, CTP, GTP and UTP

To compare the binding affinity for ATP, CTP, GTP and UTP, aptRNA (FIG. 9C) or pRNA_{wt}, (FIG. 6B) was first attached to the ATP-agarose gel. After washing with an excess amount of binding buffer, the bound RNA was then eluted by the buffer containing ATP, CTP, GTP and UTP, respectively. It was found that ATP buffer could elute the bound aptRNA or pRNA_{wt} effectively, while GTP, CTP and UTP buffer was much less efficient (FIGS. 6B and 9C).

The Central Region of phi29 pRNA is Very Similar to ATP-Binding RNA Aptamer in Both Sequence and Predicted Secondary Structure.

A chemically selected aptamer RNA has been found to be able to bind ATP (Sassanfar et al., 1993, Nature 364, 550-553) (FIGS. 7A-b). The structural basis for this ATP-binding RNA aptamer has also been elucidated by multidimensional NMR spectroscopy (Cech et al., 1996, RNA 2, 625-627; Dieckmann et al., 1996, RNA 2, 628-640; Jiang et al., 1996, Nature 382, 183-186). (FIGS. 7B-d). All ATP-binding aptamers contain a consensus sequence embedded in a common secondary structure (Cech et al., 1996, RNA 2, 625-627; Dieckmann et al., 1996, RNA 2, 628-640; Sassanfar et al., 1993, Nature 364, 550-553; Jiang et al., 1996, Nature 382, 183-186). The bases essential for ATP-binding have been identified (Sassanfar et al., 1993, Nature 364, 550-553; Jiang et al., 1996, Nature 382, 183-186). The structure of the phi29 pRNA has been investigated extensively (for review, see Guo, 2002, Prog. Nucl. Acid Res. & Mol. Biol. 72, 415-473). It would be intriguing to investigate whether the chemically selected ATP-binding RNA moiety is present in a living system. We compared the structure of ATP-binding aptamers with phi29 pRNA and found that the ATP-binding RNA aptamer is very similar to the middle part of phi29 pRNA (FIGS. 7B-c) in both sequence and structure (FIGS. 7A&B).

Infectious Virus was Produced in the Presence of the Chimeric aptRNA Harboring the ATP-Binding Moiety

To further confirm that an ATP-binding moiety is present in a pRNA molecule, the pRNA moiety with a potential for ATP-binding was replaced with an ATP-binding RNA aptamer, ATP-40-1 (Sassanfar et al., 1993, Nature 364, 550-553). A chimeric aptRNA was constructed by replacing bases 33-68 (36 bases) with the sequence of ATP-40-1 (40 bases) (Sassanfar et al., 1993, Nature 364, 550-553; Jiang et al., 1996, Nature 382, 183-186; Cech et al., 1996, RNA 2, 625-627). (FIG. 7-C). When the chimeric aptRNA was added to the phi29 in vitro assembling mixture (Lee et al., 1994, Virol, 202, 1039-1042; Lee et al., 1995, J. Virol. 69, 5018-5023) about 10⁸ infectious virus particles per milliliter were produced in the test tube (Table 1, FIG. 8). Omission of ATP or aptRNA, or the addition of RNase to the reaction mixture, failed to generate a single virus (Table 1).

ATP is Required for the Production of Infectious Virus

To establish that the activity of aptRNA is related to ATP, virus assembly using aptRNA was performed with and without the presence of ATP. When ATP was omitted from the reaction, not a single plaque was detected. Virus assembly was also inhibited by the poorly hydrolysable ATP analogue γ-S-ATP, suggesting that the aptRNA-involved viral assembly process is ATP related (Table 1).

AptRNA Bound ATP

An ATP-affinity agarose column was used to detect whether the aptRNA could bind ATP. [³H]RNA was applied to an ATP affinity column. [³H]-aptRNA was found to bind to the ATP matrix and did not run through the column (FIG. 6A-II). Additionally, aptRNA was eluted from the column with 0.004 mM ATP, suggesting that the binding of aptRNA to the column is due to specific ATP and aptRNA interaction. The 116-base rRNA negative control did not bind to the column (FIG. 6A-1).

ATP-Binding Affinity for pRNA and aptRNA

The ATP binding affinity of both RNAs were evaluated by ATP gradient elution. Free ATP (ATP_{free}) will compete with the column-bond ATP (ATP_{bound}) for binding to aptRNA or pRNA_{wt}. From the ATP gradient elution (FIG. 9), it was found that most of the bound aptRNA and pRNA_{wt} was eluted by 0.004 mM and 0.04 mM ATP_{free}, respectively.

Changing of a Single Base Essential for ATP Binding Abolished Both the ATP-Binding and Viral Assembly Activities

Nucleotide G^{con} (FIG. 7) has been shown to be highly conserved in ATP-binding RNA aptamers, and is the most critical nucleotide for ATP binding (Dieckmann et al., 1996, RNA 2, 628-640; Sassanfar et al., 1993, Nature 364, 550-553). One G corresponding to G^{con} of the aptRNA is also conserved in all the pRNAs of five different bacteriophages (Bailey et al., 1990, J. Biol. Chem. 265, 22365-22370; Chen et al., 1999, RNA 5, 805-818).

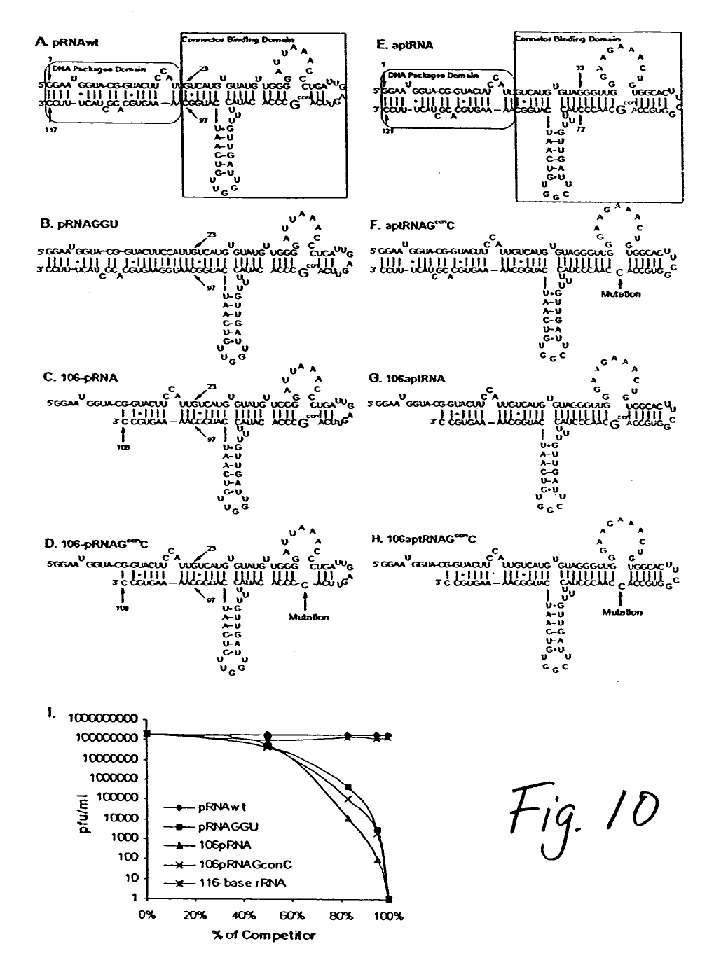

Mutation of G^{con} to C resulted in a mutant aptG^{con}C (FIG. 10F) that was not able to bind ATP (FIGS. 6A-I). This mutant was also completely inactive in virion assembly (Table 1), suggesting that the functions of ATP-binding and virion assembly are correlated. When the G^{con} mutation was introduced into the conserved G^{con} of wild type pRNA, the ATP-binding activity of the mutant pRNAG^{con}C disappeared (FIG. 6). This mutant was found to be incompetent in phi29 assembly (Table 1).

Verification of Conformation and Folding After the Change of One Single Base Essential for ATP Binding

As noted above, a single base mutation completely obliterated the activity of pRNA_{wt} and aptRNA in both ATP-binding and virion assembly. To confirm that the loss of activity in such a single base mutation is due to the change of pRNA chemistry rather than to the change in conformation or folding, competitive inhibition assays were performed (see Materials and Methods) to test whether the conformation of the mutant RNA is identical to its parental pRNA.

Two pRNAs, 106-pRNA and 106-pRNAG^{con}C (FIG. 10), were used for structural comparison. In these two pRNAs, eleven nucleotides from G¹⁰⁷ to C¹¹⁷ in the DNA translocating domain were deleted. It has previously been shown that the deletion of these eleven nucleotides did not affect the connector binding affinity of the resulting mutants (Trottier et al., 1997, J. Virol. 71, 487-494; Trottier et al., 1996, J. Virol. 70, 55-61; Garver et al., 1997, RNA 3, 1068-1079; Chen et al., 1999, RNA 5, 805-818). If the change of G^{con} to C would change the conformation or folding of the mutant pRNA, the resulting mutants 106-pRNAG^{con}C will not be able to compete with its parental pRNA_{wt} for binding to procapsid or other substrates, and thus will not be able to inhibit the parental pRNA_{wt} for procapsid binding, DNA packaging and phi29 assembly.

Competitive inhibition analysis revealed that 106-pRNAG^{con}C mutants were able to compete with pRNA_{wt} for procapsid binding and inhibit the assembly of phi29 virions (FIG. 9). Comparison of inhibition curves (FIG. 10-I) revealed that the inhibition efficiency of 106-pRNAG^{con}C is very similar to the control 106-pRNA as well as pRNAGGU, that has been shown to maintain wild type conformation in the procapsid binding domain (Chen et al., 1999, RNA 5, 805-818; Zhang et al., 1997, RNA 3, 315-322). Therefore, it can be concluded that the changing of G ^{con} to C did not cause a conformational change in the resulting mutant pRNAs in relation to procapsid binding. Competitive inhibition analysis also revealed that the inhibition profile of mutant 106-aptRNAG^{con}C is very similar to that of 106aptRNA (FIGS. 10H and G), supporting the conclusion that the changing of G^{con} to C did not cause a significant conformational change.

Conformational Changes of pRNA Induced by ATP During Packaging

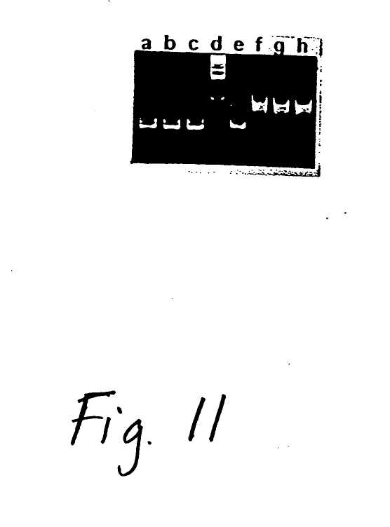

The conformation change of pRNA_{wt} was investigated in the presence and absence of ATP. ATP caused a change in the pRNA_{wt} migration rate in native gels (FIG. 11). Purified pRNA_{wt} was loaded onto an 8% native polyacrylamide gel (Chen et al., 2000, J. Biol. Chem. 275(23), 17510-17516) with increasing concentrations of ATP. A pRNA band shift was observed in the presence of ATP (lane f-h), but not observed in the absence of ATP (lane e), while the 5S rRNA control did not show any migration change either in the presence (lanes b-c) or absence (lane a) of ATP. The band with the slower migration rate was purified and shown to be fully active in DNA packaging. At the same time, control E. coli 5S rRNA did not show any migration rate change due to the presence or absence of ATP (FIG. 11).

We have previously reported that pRNA formed oligomers with slower migration rate in gel when magnesium is present (Guo et al., 1998, Mol. Cell. 2, 149-155). Chen et al., 2000, J. Biol. Chem. 275(23), 17510-17516) The formation of a band with a slower migration rate in FIG. 11 suggests that, in the presence of ATP, the conformation or oligomerization of pRNA is larger rather than smaller. This phenomenon argues against the possibility that the change of pRNA conformation is due to the depletion of ion by ATP. If that were true, the RNA should become smaller and run faster in the presence of ATP. The appearance of a broad band representing pRNA with a slower migration rate also suggests that more than one conformation of pRNA may be present, or that the pRNA/ATP complex is relatively unstable.

ATP Was Hydrolyzed to ADP and Inorganic Phosphate In A Reaction Mixture pRNA

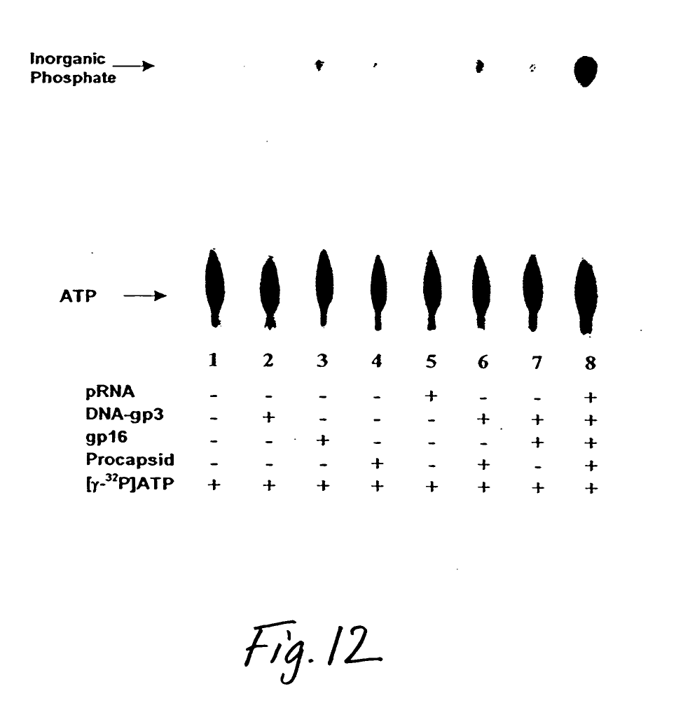

Hydrolysis of [³²P]-ATP was assayed by thin layer chromatography on a PEI-cellulose plate. Components involved in DNA packaging were mixed, alone or in combination, with [³²P]-ATP. After an incubation period of 30 minutes, the reaction mixture was applied to the PEI-plate. Results from thin layer chromatography revealed that the individual component alone or in combination without the presence of pRNA (FIG. 12), exhibited low undetectable ATPase activity. However, ATP was hydrolyzed to inorganic phosphate in the reaction including pRNA.

DISCUSSION

To secure the continuous motion of the nanomotor, at least one component should act processively to keep the motor drive continually. In bacterial virus phi29, the DNA-packaging motor is composed of the connector, gp 16 and ATP. The connector is excluded from the candidate list of processive factor, since the crystal structure of connector reveals no potential ATP-binding pocket. Gpl6 and pRNA are the only candidates for this processive factor.

Our results showed that both gp16 and pRNA are not needed to convert the finished DNA-filled capsids into infectious viruses (FIGS. 2, 3 and 4). This is comprehensible since the DNA-packaging in these particles has been completed. However, to convert the partially filled DNA-packaging intermediates into completed DNA-filled particles, fresh gp16 and ATP but not pRNA are needed (FIG. 4). This is an indication that multiple copies of fresh gp16 and ATP have to jump in to join the DNA translocating process. However, the six copies of pRNA that have already bound to the motor are sufficient to complete the DNA packaging work. In addition, pRNA were working during the DNA packaging process, since when the intermediates were treated with RNase, DNA-packaging in intermediates could not be completed and no infectious virus was produced from the intermediate (FIG. 4d). In combination with the fact that pRNA could bind ATP, it is predicted that pRNA is the processive factor in phi29 DNA packaging motor.

It has also been shown that six copies of pRNA bind to the connector (Trottier et al., 1997, J. Virol. 71, 487-494; Zhang et al., 1998, Mol. Cell. 2, 141-147, Hendrix, 1998, Cell 94, 147-150; Guo et al., 1998, Mol. Cell. 2, 149-155) that is embedded in an icosahedral protein shell that has a five-fold rotational symmetry (Simpson et al., 2000, Nature 408, 745-750; Jimenez et al., 1986, Science 232, 1113-1115). If the nanomotor indeed rotates, then the setting of the hexameric pRNA within a 5-fold symmetrical environment could constitute a mechanical apparatus with two symmetrically mismatched rings that will produce a continuous rotating force in order to drive the motor (Chen et al., 1997, J. Virol. 71, 3864-3871; Hendrix, 1978, Proc. Natl. Acad. Sci. USA 75, 4779-4783) Conformational change of molecules induced by ATP is a common phenomenon in biosystems, such as myosin, kinesin, helicase and RNA polymerase that involve motion. Our finding that ATP induced a conformational change of pRNA might boost a speculation that pRNA is part of the driving force, displaying contraction and relaxation as proposed previously (Chen et al., 1997, J. Virol. 71, 3864-3871).

Mutation studies of pRNA_{wt} and aptRNA have revealed that, within each pRNA_{wt} or aptRNA group, ATP-binding affinity is correlated to phi29 virion assembly (Table 1). However, outside the group, this correlation could not apply. For example, the ATP-binding affinity of aptRNA is stronger than pRNA_{wt}, but the viral assembly activity of aptRNA is not higher than pRNA_{wt} (FIG. 8D). Three possibilities might explain this discrepancy. First, the binding of pRNA to the connector is the rate determining step in phi29 DNA packaging and assembly. A 29-base change in the connector-binding domain of aptRNA might somehow alter its structure and thus hamper the connector binding affinity. As shown in FIG. 8D, the concentration requirement to reach a 50% plateau of the assembly curve for aptRNA is higher than for pRNA_{wt}. This is an indication that the binding affinity (Kₐ) of aptRNA/connector complex is lower than that of pRNA_{wt}/connector complex. Second, although the chemically selected ATP-binding aptamer is an excellent molecule for ATP binding, it might not be, after all, the best candidate in nature for ATP hydrolysis if such hydrolysis does occur. Third, too high a binding affinity to the substrate does not signify a good enzyme, since this enzyme will not be dissociated from its substrate easily. Such dissociation might be critical for the turnover in pRNA/ATP interaction in phi29 assembly.

Here we found that the putative ATP-binding site in pRNA resides within a region interacting with the connector. The significance for such ATP/pRNA binding remains to be investigated. One possible implication is that ATP binding to pRNA provides a special structure in the assembly of the packaging machinery. Another possible implication is that alternative binding and release of ATP from pRNA could induce a conformational change of pRNA that in turn rotates the connector.

### Example II

### Construction of a Controllable 30-nm Nanomotor Driven by a Synthetic ATP-Binding RNA

Experimental Procedures

Synthesis of aptRNA

AptRNA (FIG. 7C) was synthesized both chemically and enzymatically. With the chemical method, an additional ligation step was used to synthesize the 121-base aptRNA from smaller synthetic RNA oligonucleotides. With the enzymatic method, RNA was synthesized with T7 RNA polymerase by run-off transcription and purified from a polyacrylamide gel. The sequences of both plasmids and PCR products were confirmed by DNA sequencing. No difference in DNA-translocation and viral assembly activity was found with RNA from both methods.

In Vitro Construction of the Nanomotor and Testing of Motor Function By Its Ability To Produce Infectious phi29 Virion.

Procapsids and gp16, as well as the phi29 structural proteins gp9, gp 11 and gp12 were purified from products of genes that were cloned into plasmid. pRNA enriched procapsids were synthesized as in Example I. The pRNA-enriched procapsids were then mixed with purified gp16, DNA, and ATP to complete the DNA packaging reaction (the first round, DNA packaging). After 30 minutes, gp 11, gp12, and gp9, gp13, and fresh gp16 were added to the DNA packaging reactions in the second round (phage assembly) to complete the assembly of infectious virions, which were assayed by standard plaque formation.

Testing For Turning Off and On of the Motor Function

The motor was turned off by the addition of ATP analogue, and the DNA-packaging intermediates with partially packaged DNA were isolated due to the halting of the motor. The turned off motor was turned on again by the addition of ATP and assayed for the production of infectious virion. DNA-packaging intermediates were isolated and converted into infectious phage as in Example I.

ATP-Binding Assay for pRNA With ATP-Agarose Affinity Column

ATP binding of aptRNA and related molecules was accomplished as in Example I.

Gel Shift Assay

Purified aptRNA was loaded onto an 8% native polyacrylamide gel with an increasing amount of ATP. A 5S rRNA was used as a control.

Determination of Apparent Dissociation Constants (K_{D,app}) for aptRNA/ATP Complex.

The K_{D,app} for RNA/ATP interaction was determined by the methods of isocratic elution and ATP gradient elution. The isocratic elution method was used to measure the K_{D,app} for ATP that immobilized on agarose (ATP_{bound}), while the method of ATP gradient elution was to measure the K_{D,app} for free ATP (ATP_{free}).

Isocratic elution. [³H]aptRNA was applied to a column (0.55 cm in diameter) packed with ATP-C-8 affinity agarose (2.7 ml) and eluted with binding buffer. Fractions (2 ml) were collected and subjected to scintillation counting. KD,app was determined with the equation: K_{D,app}=[L]×(Vₜ−V₀)/(Vₑ−V₀) where [L] is the concentration of ATP immobilized on agarose (1.7 mM), Vₜ is the volume of the column (2.7 ml), V₀ is the void volume of the column (2.09 ml), and Ve is the volume needed to elute the RNA (32 ml). The K_{D,app} for aptRNA interacting with the ATP_{bound} was determined to be 0.035 mM.

ATP gradient elution. [³H]aptRNA was applied to a column (0.55 cm in diameter) packed with ATP-C-8 affinity agarose (0.8 ml) and eluted with a 2 ml step-up gradient with a specified concentration of ATP in binding buffer. Fractions were collected and subjected to scintillation counting. The K_{D,app} for the complex of aptRNA/ATP_{free} is around 0.004 mM.

results

Infectious Viruses Were Produced in the Test Tube Using the Artificial aptRNA

The gene coding for the three bacterial virus phi29 protein components gp7, gp8 and gp10 that are needed for building a functional virus were cloned into plasmid and transformed into E. coli cells The particles assembled in E. coli were similar to phi29 procapsids. The purified particles from E. coli were then incubated with the synthetic aptRNA, which automatically bound to the particles. In the presence of ATP, this RNA could power a motor to rotate and move the 19 Kbp-phi29 genomic DNA into the protein shell to produce infectious viral particles in vitro with a titer of 108 infectious virus particles per milliliter (Table 2, FIG. 8). Omission of ATP or aptRNA or the addition of RNase to the reaction mixture failed to generate a single virus (Table 2).

TABLE 2
Production of Infectious virus with aptRNA and ATP

| | Virus |
|---|---|
| Components | produced |

| AptRNA | ATP | RNase | γ-S-ATP | (pfu/ml) |
|---|---|---|---|---|
| + | + | − | − | 2 × 10⁸ |
| − | + | − | − | →0 |
| + | − | − | − | →0 |
| + | + | + | − | →0 |
| + | + | − | + | →0 |

AptRNA Bound ATP

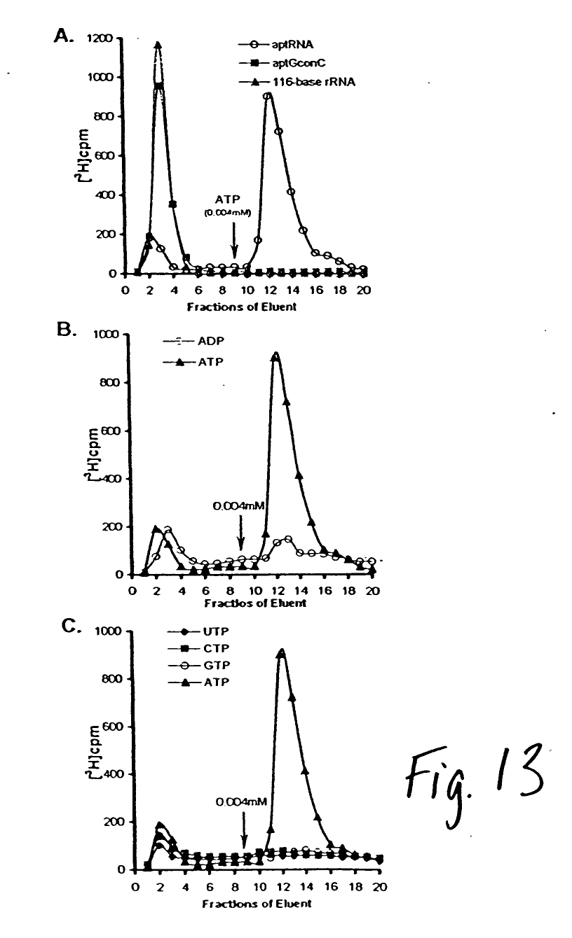

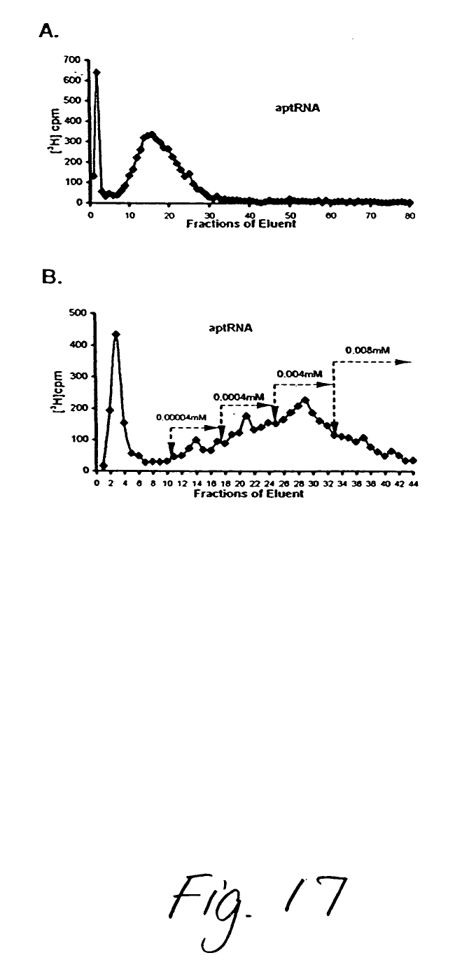

An ATP-affinity agarose column was used to detect whether the aptRNA could bind ATP. [³H]RNA was applied to an ATP affinity column. Most [³H]aptRNA was found to bind to the ATP matrix and did not run through the column (FIG. 13). Additionally, aptRNA was eluted from the column with 0.004 mM ATP, suggesting that the binding of aptRNA to the column is due to specific ATP and aptRNA interaction. AptRNA was not eluted by ADP, UTP, CTP or GTP (FIG. 13B,C). The 116-base rRNA negative control did not bind to the column (FIG. 13A). The K_{D,app} for the RNA/ATP interaction was determined to be 0.035 mM for resin-bound ATP and 0.004 mM for free ATP (FIG. 17). The finding of a difference in the K_{D,app} determined via these two methods is not surprising because the C-8 linkage of ATP to agarose might hamper the RNA/ATP interaction that involves a three-dimensional contact. Furthermore, it is possible that only a certain fraction of ATP_{bound}in the gel is accessible to aptRNA.

Comparison of aptRNA Binding Affinity to ATP and ADP

In bio-systems, energy is derived from the hydrolysis of ATP to ADP. It would be interesting to know whether aptRNA can discriminate ATP from ADP. Both ATP and ADP-affinity agarose columns were immobilized with ATP or ADP, respectively, and attachments through the C8 position were used to compare their binding affinity for aptRNA. As noted earlier, aptRNA could attach to an ATP-affinity agarose column. However, when aptRNA was applied to the ADP-column, most of the aptRNA did not bind to the column but passed through, appearing only in the first several fractions of the elution. When the ADP column was eluted with 4 mM ADP or ATP, the elution of aptRNA from the ADP column was very low. The concentration used here was 1000-fold higher than that used for the ATP column, indicating that the binding affinity of aptRNA to ADP was much lower than that of ATP.

Other approaches for affinity comparison were also made. [³H]aptRNA was applied to the ATP-affinity agarose column first, then eluted by ATP and ADP, respectively. Comparison of the elution profiles by ATP and ADP revealed that most of the bound aptRNA was eluted by 0.004 mM ATP. However, in spite of an expected higher affinity for free ADP than for immobilized ADP, very little aptRNA was eluted by ADP (FIG. 13B), even with an ADP concentration as high as 5 mM, supporting the conclusion that the binding affinity of aptRNA to ADP was much lower than that of ATP.

Comparison of AptRNA Binding Affinity for ATP, CTP, GTP and UTP

AptRNA was first attached to the ATP-agarose gel. After washing with an excess amount of binding buffer, the bound RNA was then eluted by buffers containing ATP, CTP, GTP and UTP, respectively. It was found that the ATP buffer could elute the bound aptRNA effectively, while the GTP, CTP and UTP buffers were much less efficient (FIG. 13C).

Changing of A Single Base Essential for ATP Binding Abolished Both the ATP-Binding and Viral Assembly Activities

The structural basis for ATP-binding RNA aptamers has also been clarified by multidimensional NMR spectroscopy. All ATP-binding aptamers contain a consensus sequence embedded in a common secondary structure and the bases essential for ATP-binding have been identified. Nucleotide G^{con} (Example I, FIG. 7C) has been shown to be highly conserved in ATP-binding RNA aptamers and is the most critical nucleotide for ATP binding.

Mutation of G^{con} to C resulted in a mutant aptG^{con}C (Example I, FIG. 7C) that was not able to bind ATP (FIG. 13A). This mutant was also completely inactive in virus assembly (Table 3), suggesting that the functions of ATP-binding and virus assembly are correlated. By structural analysis, in addition to competition and inhibition with binomial distribution analysis, it was confirmed that the incompetence of such mutant aptRNA in motor driving is due to a change in chemistry rather than structure.

TABLE 3
Activities of aptRNA and Mutant

| | | | Virus produced |
|---|---|---|---|
| RNAs | Mutation | ATP-binding | (pfu/ml) |
| aptRNA | none | + | 10⁸ |
| aptG^{con}C | G^{con →}C | − | →0 |
| 116-base rRNA | N/A | − | →0 |

ATP Is Required For the Production of Infectious Virus

To establish that the activity of aptRNA is related to ATP, virus assembly using aptRNA was performed with and without the presence of ATP. When ATP was omitted from the reaction, not a single plaque was detected. Virus assembly was also inhibited by the poorly hydrolysable ATP analogue γ-S-ATP, suggesting that the aptRNA-involved viral assembly process is ATP related (Table 2).

Conformational Changes of the ATP-Binding RNA Induced By ATP

In the mechanism of the movement of muscle, alternative binding and release of ATP induces a conformational change of the muscle to produce a transition. Does aptRNA move by conformational change induced by ATP? The change in conformation of the ATP-binding RNA was investigated both in the presence and absence of ATP using a gel shift assay. Purified ATP-binding RNA was loaded onto a native gel with increasing concentrations of ATP. ATP caused a change in the RNA migration rate in native gels (FIG. 11). The ATP-binding RNA was observed to migrate slower when ATP was present. A band shift of ATP-binding RNA was observed in the presence of ATP (lane f-h), but not observed in the absence of ATP (lane e), while the 5S rRNA control did not show any migration change either in the presence (lanes b-c) or absence (lane a) of ATP. The band with the slower migration rate was purified and shown to be fully active in DNA packaging. At the same time, a control E. coli 5S rRNA did not show any migration rate change in the presence or absence of ATP (FIG. 11).

It has previously been reported that pRNA formed oligomers with a slower migration rate in gel when magnesium was present. The formation of a band with a slower migration rate in FIG. 11 suggests that, in the presence of ATP, the conformation or oligomerization of pRNA is larger rather than smaller. This phenomenon argues against the possibility that the change of ATP-binding conformation is due to the depletion of an ion by ATP, but in favor of a speculation that ATP induces RNA conformational changes. If that were true, the RNA should become smaller and run faster in the presence of ATP. The appearance of a broad band representing ATP-binding RNA with a slower migration rate also suggests that more than one conformation of ATP-binding RNA may be present, or that the RNA/ATP complex is relatively unstable.

ATP Was Hydrolyzed to ADP and Inorganic Phosphate In A Reaction Mixture With aptRNA

To assay for ATPase activity, components involved in DNA packaging were mixed alone, or in combination, with [³²P]ATP. Results from thin layer chromatography revealed that the individual components alone, or in combination without the presence of aptRNA (FIG. 12), exhibited low undetectable ATPase activity. However, ATP was hydrolyzed to inorganic phosphate in the reaction including aptRNA.

Motor Could be Turned Off by EDTA, γ-S-ATP and RNase

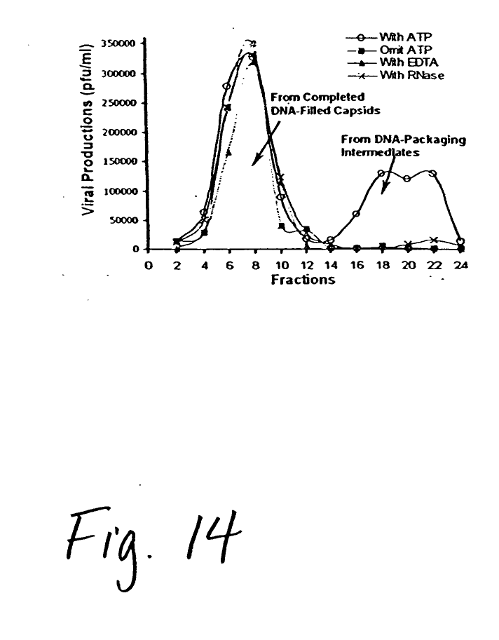

One of the important issues in constructing a viable molecular motor or shuttle involves how to switch it on and off. It was shown that this DNA-packaging motor could be turned off with the addition of EDTA, RNase (FIG. 14), or poorly hydrolyzable ATP analogues, such as γ-S-ATP (FIG. 2, Example 1).

The Turned-Off Motor Can be Started Again By ATP Or Magnesium, But Is Irreversible If Shut Off By RNase

A usable motor must be able to run again after being shut off. To test whether the stationary motor turned off by EDTA, RNase or γ-S-ATP could be switched on again, the intermediates containing blocked motors were isolated. Intermediates were separated from free DNA, finished DNA-filled capsids or empty procapsids by sucrose gradient sedimentation as in Example I. ATP, gp16, gp 11/12 and gp9 were added to each of those fractions from the sucrose gradient that contained DNA-packaging intermediates, and assayed for the production of infectious virus. The production of infectious virus from completed DNA-filled particles was used as an indicator in testing the motor function in DNA packaging.

It was found that nanomotors turned off by γ-S-ATP were turned on again by ATP, since the DNA-packaging intermediates blocked by γ-S-ATP could be converted into matured infectious virion by the addition of gp 16 and ATP as well as the neck protein gp 11/gp12 and the tail protein gp9 (FIG. 14). The addition of ATP allowed the packaging of the entire viral DNA genome to be completed. The reactivation of the stationary nanomotor by ATP was further confirmed by direct observation of RNA rotation.

When EDTA was used to turn off the nanomotor, further analysis revealed that magnesium could turn it back on. However, a stationary nanomotor turned off by RNase was irreversible (FIG. 14).

The Nanomotor Could Be Turned On and Off By gp16

As shown in Example I (FIGS. 4a and 4b), it was found that the candidate of the processive factor in this DNA-packaging motor is pRNA, while gp16 is a transient distributive factor in motor function. AptRNA functions as pRNA in the nanomotor. That is, aptRNA is an integrated solid part of the nanomotor, but gp16 is not. Without the addition of fresh gp16, not a single infectious virus particle was produced from the intermediates. This indicates that additional fresh gp16 is needed to complete assembly and that alternate gp16 molecules must have been involved in the DNA-packaging process. To repeat, gp 16 is not a fixed solid part of the nanomotor, and the function of gp 16 is contributive.

Packaged DNA Was Released From the Protein Shell In the Presence of EDTA at Low pH or High Temperature

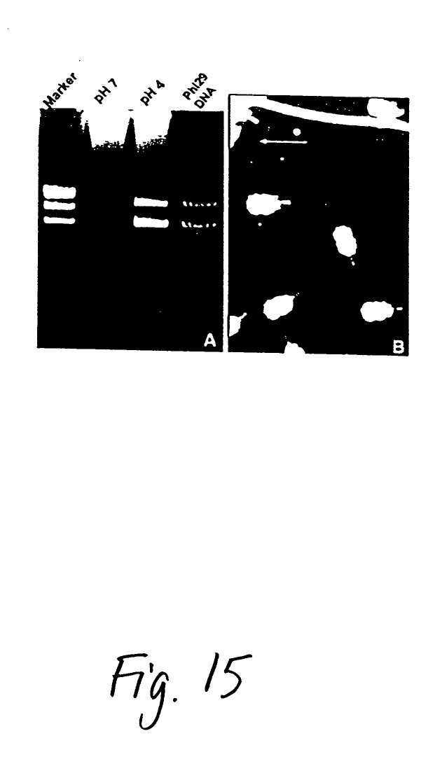

To determine the conditions for the reverse function of the nanomotor, the completed DNA-filled particles or infectious mature virions were treated with different pH, temperature and chemicals. The phi29 particles were contacted with buffers having pH 7 and pH 4 (lane c), then neutralized to pH 7, digested with the restriction enzyme EcoRI, and subjected to gel electrophoresis. FIG. 15 shows the pH 7 (lane b) and pH 4 (lane c) samples. It was found that in the presence of EDTA, the packaged DNA was released from the protein shell at pH 4 (FIG. 15), or at 75° C., but not at pH 7. DNA discharge is a passive motion process since no ATP is needed for such translocation.

Formation of the Ordered Structural Arrays

Due to the limitation in size, it is extremely difficult to detect, observe and build a structure using nano-parts. Formation of ordered structural arrays will greatly facilitate the application of nano-parts, such as in the manufacture of computer chips.

It was found that the in vitro synthesized nanomotor and motor parts formed a hexameric array, pentagonal particles and tetragonal arrays, depending on the condition and the number of parts present.

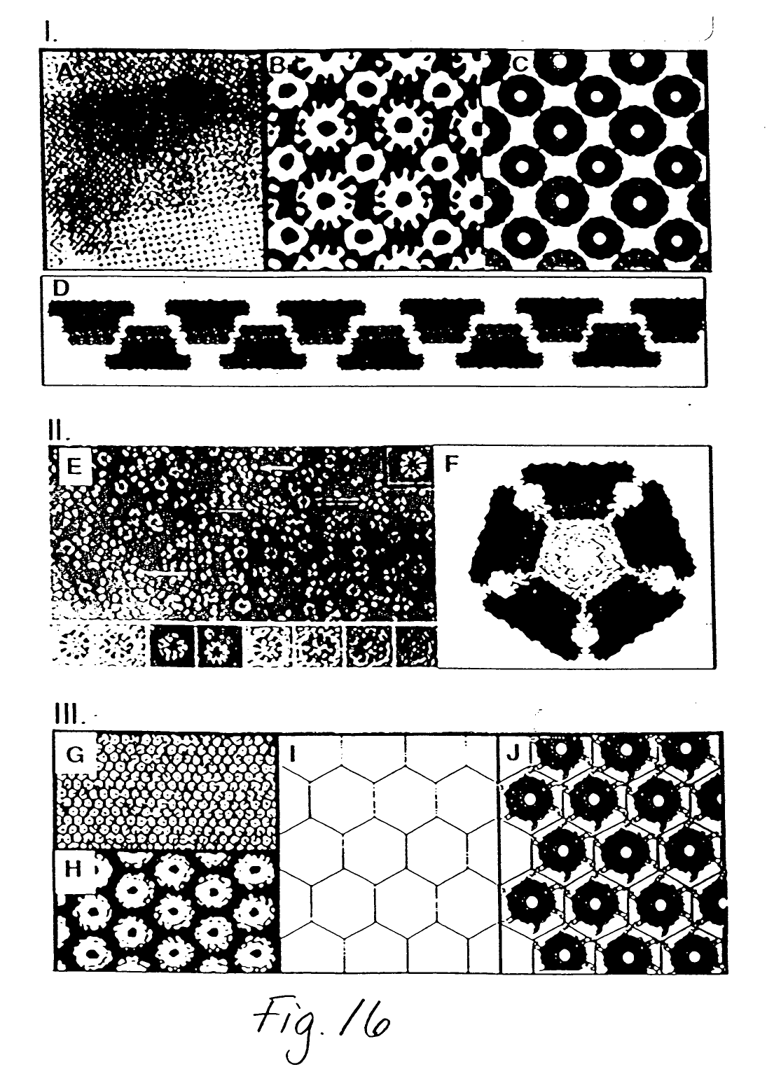

In 3M NaCl, the purified recombinant connector, composed of 12 subunits of gp10 protein, formed a well-ordered tetragonal array. Since the connector is a trapezoid-shaped cone, alternating facing-up and facing down arrangements facilitated the formation of the tetragonal array (FIG. 16-I).

When six pRNAs were bound to the connector, the tetragonal arrays disappeared immediately. Rosettes containing five complexes composed of connector and hexameric RNA were formed with the RNA located at the center of the pentagonal rosette (FIG. 16-II).

When an additional protein gp 11 was added to the connector, a hexagonal array instead of tetragonal arrays was detected (FIG. 16-III). The formation of the hexagonal array is due to the six-fold symmetry of the 12-subunit connector and the filling up of the narrow end of the trapezoid/cone-shape by the addition of six copies of pRNA and 12 copies of gp11 after an interaction with a hexameric RNA.

Up to 120 Nonspecific Bases Can be Extended From the 3′-End of aptRNA Without Hindering the Function of the Nanomotor

To investigate whether additional burden can be imposed to the RNA, both the 3′ and 5′-ends of the aptRNA were extended with variable length. It was found that the 5′-end is not extendable, since a single base addition will render the RNA incompetent to drive the motor. However, up to 120 bases can be added to the 3′-end of the aptRNA without a significant interference of the motor function. Such addition includes the labeling with biotin, pCp, DIG and phosphate.

Discussion

The construction of a practical molecular shuttle requires a careful consideration of guiding the direction of motion, controlling the on-off status and speed, as well as the loading and unloading of cargo.

It was found that the direction of the DNA-packaging motor could be guided by adjusting the pH, the temperature or by the addition or omission of EDTA or ATP.

The nanomotor can be turned off by EDTA, γ-S-ATP, or RNase. Although the inactivation of the nanomotor by RNase was irreversible, the EDTA and γ-S-ATP effect can be negated by the addition of magnesium and/or ATP, respectively. This is an indication that the nanomotor inactivated by γ-S-ATP could be turned on by ATP, and that the nanomotor turned-off by EDTA could be turned on again by magnesium.

Gp 16 can be used to control the running of the nanomotor, since a continuous supply of fresh gp 16 is needed to keep the motor functioning. The control of ATP concentration, acting as a fuel supply, can serve as a means of controlling the speed of movement.

The loading process requires the coupling of cargo to the shuttle. The 120 bases extended from the 3′-end could serve as a tool for loading cargo. This can be achieved by attaching the cargo to a DNA that is complementary to the sequence at the 3′ end of the aptRNA. The formation of ordered structural arrays or particles will facilitate the construction of nanomachines. All this suggests that this DNA-packaging motor is a candidate component for use in the construction of nanodevices. This motor, expected to be a rotary machine with a mechanism similar to phi29 DNA-packaging motor that rotates in 12° increments, has been solved by mathematical simulation and direct observation.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A molecular rotary nanomotor comprising, as structural components:
a gp10 connector protein;
a gp8 capsid protein; and
a non-naturally occurring pRNA;
wherein the structural components are associated with one another to form a nanoscale structure that effects translocation of a polynucleotide in the presence of a gp16 protein, ATP and Mg^{++.}

2. The molecular nanomotor of claim 1 wherein the non-naturally occurring pRNA is one that folds into a structure similar to that of naturally occurring phi29 pRNA (SEQ ID NO: 2).

3. The molecular nanomotor of claim 1 further comprising a protein gp7.

4. The molecular nanomotor of claim 1 wherein the translocation activity can be reversibly stopped by contacting the nanomotor with a metal chelating agent, contacting the nanomotor with a nonhydrolyzable ATP analogue, or depriving the nanomotor of a source of gp16 protein, ATP or Mg⁺⁺.

5. An isolated molecular nanomotor comprising as structural components:
a connector protein;
a capsid protein; and
a pRNA;
wherein the structural components are associated with one another to form a nanoscale structure that effects translocation of a polynucleotide in the presence of ATP and Mg⁺⁺, and wherein the pRNA binds ATP and drives the rotational motion of the nanomotor.

6. The isolated molecular nanomotor of claim 5 wherein the pRNA is selected from the group consisting of SF5 pRNA (SEQ ID NO: 5), B103 pRNA (SEQ ID NO: 6), M2/NF pRNA (SEQ ID NO: 7) and GA1 pRNA (SEQ ID NO: 8).

7. The isolated molecular nanomotor of claim 5 wherein the pRNA folds into a structure similar to that of naturally occurring pRNA from SF5, B 103, M2/NF or GA 1.

8. The isolated molecular nanometer of claim 5 wherein the pRNA is a non-naturally occurring pRNA.

9. A method for translocating a polynucleotide comprising:
providing a molecular nanomotor having a nanoscale structure according to claim 1; and
contacting the nanoscale structure with a gp16 protein, ATP and Mg⁺⁺under conditions to translocate the polynucleotide.

10. The method of claim 9 wherein the contacting step further comprises contacting the nanoscale structure with polyethylene glycol.

11. The method of claim 9 further comprising contacting the nanoscale structure with a chelating agent or a nonhydrolyzable ATP analogue to reversibly stop translocation of the polynucleotide.

12. The method of claim 11 wherein the chelating agent is EDTA.

13. The method of claim 11 wherein the nonhydrolyzable ATP analogue is γ-S-ATP.

14. A method for translocating a polynucleotide comprising:
providing a molecular nanomotor having a nanoscale structure according to claim 5; and
contacting the nanoscale structure with a gp16 protein, ATP and Mg⁺⁺under conditions to translocate the polynucleotide.

15. The method of claim 14 wherein the contacting step further comprises contacting the nanoscale structure with polyethylene glycol.

16. The method of claim 14 further comprising contacting the nanoscale structure with a chelating agent or a nonhydrolyzable ATP analogue to reversibly stop translocation of the polynucleotide.

17. The method of claim 16 wherein the chelating agent is EDTA.

18. The method of claim 16 wherein the nonhydrolyzable ATP analogue is γ-S-ATP.

19. A nanodevice comprising the molecular nanomotor of claim 1.

20. A nanodevice comprising the molecular nanomotor of claim 5.
